SEQUENCE LISTING (9 sequences)

SEQ ID NO: 1 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 194 aa, Homo sapiens
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 3 - protein, 260 aa, Homo sapiens
MAKDNSTVRCFQGLLIFGNVIIGCCGIALTAECIFFVSDQHSLYPLLEATDNDDIYGAAWIGIFVGICLFCLSVLGIVGIMKSSRKILLAYFILMFIVYAFEVASCITAATQQDFFTPNLFLKQMLERYQNNSPPNNDDQWKNNGVTKTWDRLMLQDNCCGVNGPSDWQKYTSAFRTENNDADYPWPRQCCVMNNLKEPLNLEACKLGVPGFYHNQGCYELISGPMNRHAWGVAWFGFAILCWTFWVLLGTMFYWSRIEY

SEQ ID NO: 4 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 5 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 6 - DNA, 25 bp, synthetic construct
  source 1..25: mol_type = other DNA
ggcagcagctgcttcggatccacac

SEQ ID NO: 7 - DNA, 25 bp, synthetic construct
  source 1..25: mol_type = other DNA
cgctgccaaggctgtgggcaaggtc

SEQ ID NO: 8 - DNA, 25 bp, synthetic construct
  source 1..25: mol_type = other DNA
  modified_base 1..2: mod_base = OTHER; note = Locked nucleic acid
  modified_base 24..25: mod_base = OTHER; note = Locked nucleic acid
cacttacaggaatatgcataacatg

SEQ ID NO: 9 - DNA, 22 bp, synthetic construct
  source 1..22: mol_type = other DNA
  modified_base 1..2: mod_base = OTHER; note = Locked nucleic acid
  modified_base 21..22: mod_base = OTHER; note = Locked nucleic acid
ggaagcgaaaatgaaattgact

(19) United States
(12) Patent Application Publication
(10) Pub. No.: US 2025/0059545 A1
(43) Pub. Date: Feb. 20, 2025

(54) METHODS FOR TREATING BLADDER CANCER

(71) Applicant: HOSPITAL ESPAÑOL AUXILIO MUTUO DE PUERTO RICO, INC., San Juan, PR (US)

(72) Inventor: Eduardo I. CANTO REPETTI, San Juan, PR (US)

(21) Appl. No.: 18/935,823

(22) Filed: Nov. 4, 2024

Related U.S. Application Data
(63) Continuation of application No. PCT/US2023/066612, filed on May 4, 2023.
(60) Provisional application No. 63/408,181, filed on Sep. 20, 2022.
(60) Provisional application No. 63/408,180, filed on Sep. 20, 2022.
(60) Provisional application No. 63/338,676, filed on May 5, 2022.
(60) Provisional application No. 63/338,671, filed on May 5, 2022.

(51) Int. Cl. C12N 15/113; A61K 35/742; A61P 35/00; C07K 16/24; C12N 5/00; C12N 5/071; C12N 5/09; C12Q 1/6886; G01N 33/50; G01N 33/68

(52) U.S. Cl. CPC C12N 15/1136; A61K 35/742; A61P 35/00; C07K 16/244; C12N 5/0018; C12N 5/0685; C12N 5/0693; C12Q 1/6886; G01N 33/5023; G01N 33/6866; C12N 2310/11; C12N 2501/24; G01N 2333/4703; G01N 2333/54; G01N 2333/57; G01N 2800/52

(57) ABSTRACT

Disclosed herein is a method for selecting a subject for bladder cancer therapy based on determining from the sample from a subject with bladder cancer, the presence, absence, or level of a biomarker that correlates with interleukin 18 binding protein (IL-18 BP) expression by bladder cells from a subject. Also disclosed herein are compositions comprising an effective amount of at least one of interleukin 18 (IL-18) and an inhibitor of IL-18 binding protein (IL-18 BP), wherein the inhibitor causes at least one of reduces binding of IL-18 BP to IL-18 and reduces IL-18 BP levels. The compositions may be used in methods for treating bladder cancer, comprising administering to a subject in need thereof an effective amount of at least one of IL-18, an inhibitor of IL-18 BP, an antagonist of an activator of IL-18 BP, and an agonist of a down-regulator of IL-18 BP.

### 1. CROSS-REFERENCE TO RELATED APPLICATIONS

This application claims the benefit of U.S. Provisional Patent Application No. 63/408,181, filed Sep. 20, 2022, U.S. Provisional Patent Application No. 63/408,180, filed Sep. 20, 2022, U.S. Provisional Patent Application No. 63/338,676, filed May 5, 2022, and U.S. Provisional Patent Application No. 63/338,671, filed May 5, 2022, which are incorporated herein by reference in their entirety.

### 2. BACKGROUND

Bladder cancer is estimated to afflict about 1.5 million people globally, with roughly 500,000 new cases and about 200,000 deaths every year. Five-year survival rates are about 70-75% in the developed world. Part of this relatively high survival rate stems from a well-developed standard of care for early-stage, non-invasive bladder cancers.

The standard of care involves an intravesicular biopsy, which may be performed simultaneously with resection of the tumor. Roughly two to six weeks later, treatment with bacillus Calmette-Guérin (BCG) begins, which generally involves one intravesicular administration per week for five to six weeks of BCG into the patient's bladder. Though not to be bound by theory, the current understanding among workers in the field is that BCG induces an immune response, attracting neutrophils that produce cytokines which attract antigen-presenting cells (APCs) and macrophages. APCs and macrophages produce IL-18 and IL-12, which activate helper T cells, natural killer cells, and additional macrophages to attack BCG. Bladder cancer cells are also attacked by the BCG-induced immune response, both directly by the cells and by production of interferon gamma, in what could be considered collateral damage in the immune system's attack on BCG. Interferon gamma can also be referred to as IFNgamma, IFNg, or IFNγ. Efficacy of BCG therapy is generally assessed by biopsy about six to twelve weeks after completing the course of BCG treatment.

Unfortunately, roughly 30-50% of patients are not responsive to BCG therapy.

Although non-BCG therapies are well known and are used as a second line of defense for BCG non-responders, the current standard of care subjects BCG non-responders to roughly 12-18 weeks of ineffective treatment, in which time the patient's bladder cancer could progress, perhaps necessitating more extreme therapeutic actions than if the cancer were effectively treated earlier. Even if the patient's bladder cancer does not progress, the patient would spend unnecessary time undergoing unpleasant intravesicular procedures. Further, BCG does pose a slight risk of systemic infection, which for non-responders, is not accompanied by the strong potential benefit of treating the patient's bladder cancer.

Also, subjecting a patient to an ineffective treatment is an inefficient use of health care system resources, such as clinical staff time, BCG formulations, etc., imposing unnecessary financial costs on one or more of a governmental payor, a charitable or other NGO payor, an insurance provider, or the patient and/or his/her family.

It would be desirable to stratify patients suffering from non-invasive bladder cancer into BCG responders and BCG non-responders prior to the start of BCG therapy. It would also be desirable for patients suffering from non-invasive bladder cancer who would otherwise not respond to BCG therapy to receive an additional intervention that would render them responsive to BCG therapy.

### 3. SUMMARY

This disclosure relates to a method for determining treatment for bladder cancer, comprising providing bladder cells from a subject with bladder cancer; culturing at least a portion of the cells with IFNγ and/or culturing at least a portion of the cells with IFNγ and TNFα, each culture maintained for a period of time sufficient to induce expression of interleukin 18 binding protein (IL-18 BP) and/or IL-18 by the cells; determining expression level of IL-18 BP and uroplakin 1B, by the cells cultured in b); determining the ratio of expression level of IL-18 BP to uroplakin 1b for cells cultured with IFNγ (IL-18BP:uroplakin 1b IFNγ) and/or for cells cultured with IFNγ and TNFα (IL-18BP:uroplakin 1b IFNγ+TNFα); wherein a IL-18BP:uroplakin 1b IFNγ of about 6.0 or greater and/or a IL-18BP:uroplakin 1b IFNγ+TNFα of about 40 or lower indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy. As indicated above, interferon gamma can also be referred to as IFNgamma, IFNg, or IFNγ. Tumor necrosis factor alpha can also be referred to as TNFalpha, TNFα or TNFα. Uroplakin 1B can also be referred to as Uroplakin 1b, UPK1B, or UPK1b. IL-18BP can also be referred to as IL-18BPa.

The sample is preferably from a bladder biopsy, preferably a bladder biopsy that includes urothelium and bladder cancer cells.

For example, the value for IL-18BP:uroplakin 1b IFNγ indicating a need for therapy is about 6.0 or greater, about 8.0 or greater, about 10.0 or greater, about 12.0 or greater, about 14.0 or greater, about 16.0 or greater, about 18.0 or greater, about 20.0 or greater, or about 22.0 or greater.

For example, the value for IL-18BP:uroplakin 1b IFNγ+TNFα indicating a need for therapy is about 40 or lower, about 36 or lower, about 32 or lower, about 28 or lower, about 24 or lower, about 20 or lower, about 16 or lower, about 12 or lower, about 8 or lower, or about 4 or lower.

For example, the culture period in b) is selected from at least about 12 hours, at least about 24 hours, at least about 36 hours, at least about 48 hours, at least about 60 hours, at least about 72 hours, at least about 84 hours, or at least about 96 hours.

For example, the expression level in c) comprises determining a protein level.

For example, the expression level in c) comprises determining a mRNA level.

For example, IL-18BP:uroplakin 1b IFNγ and IL-18BP:uroplakin 1b IFNγ+TNFα, is determined.

For example, the inhibitor, the antagonist, or the agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP expression; agonists of a down-regulator of IL-18 BP transcription; a polynucleotide, e.g, for gene therapy, to reduce expression of IL-18 BP by non-cancerous urothelial cells, bladder cancer cells, or both: a polynucleotide, e.g, for gene therapy, to increase expression of IL-18 by non-cancerous urothelial cells, bladder cancer cells, or both (e.g., an mRNA encoding IL-18): a polynucleotide, e.g., for gene therapy, to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

For example, the inhibitor of IL-18BP is an anti-IL18BP antibody.

The method can further comprise administering the therapy to a subject with bladder cancer.

For example, the therapy is administered by intravesical administration to the bladder.

For example, the bladder can be heated.

For example, at least one of the inhibitor, antagonist, and agonist reduces binding of IL-18 BP to IL-18 in the bladder or reduces IL-18 BP levels in the bladder.

For example, the administering comprises intravesicular administration of an ASO complementary to an RNA encoding IL-18 BP isoform A (IL-18 BPa), to an RNA encoding IL-18 BP isoform C (IL-18 BPc), or to an RNA encoding the activator of IL-18 BP.

For example, the ASO comprises a sequence that, upon complementation of the RNA encoding IL-18 BP or the activator thereof by the ASO, causes at least one of reduces translation of the RNA and alters splicing of the RNA encoding IL-18 BP, thereby reducing the yield of IL-18 BPa and/or IL-18 BPc.

For example, bacillus Calmette-Guérin (BCG) is be administered to the subject in need thereof; or wherein the subject in need thereof has received or will receive BCG. For example, the therapy comprises BCG and at least one of interleukin 18 (IL-18), an inhibitor of IL-18 BP, an antagonist of an activator of IL-18 BP, and an agonist of a down-regulator of IL-18 BP.

For example, the BCG is administered by intravesical administration to the bladder.

For example, the IL-18, the inhibitor, the antagonist, and/or the agonist is/are administered essentially simultaneously with the BCG.

For example, the BCG therapy is a primary BCG therapy or a maintenance BCG therapy.

For example, the subject is identified as being non-responsive to BCG monotherapy prior to administering the IL-18, the inhibitor, the antagonist, and/or the agonist.

For example, the inhibitor of IL-18 BP reduces the binding of IL-18 BP isoform A (IL-18 BPa) and/or IL-18 BP isoform C (IL-18 BPc) to IL-18 and/or reduces levels of IL-18 BPa and/or IL-18 BPc.

For example, the non-BCG therapy is selected from the group consisting of chemotherapy, radiation therapy, immunotherapy, cystectomy, and combinations thereof.

This disclosure relates to a composition, comprising: an effective amount of interleukin 18 (IL-18), an inhibitor of IL-18 binding protein (IL-18 BP), an antagonist of an activator of IL-18 BP, and/or an agonist of a down-regulator of IL-18 BP, and a pharmaceutically-acceptable excipient.

For example, the composition is formulated for intravesical administration to the bladder.

For example, the inhibitor, antagonist, or agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

For example, the composition comprises an effective amount of bacillus Calmette-Guérin (BCG).

This disclosure relates to a kit, comprising: instructions for performing the method of any one of the preceding claims, and a composition comprising an effective amount of at least one of interleukin 18 (IL-18), an inhibitor of IL-18 binding protein (IL-18 BP), an antagonist of an activator of IL-18 BP, and an agonist of a down-regulator of IL-18 BP, and a pharmaceutically-acceptable carrier.

For example, the inhibitor reduces binding of IL-18 BP to IL-18 in the bladder, reduces IL-18 BP levels in the bladder, and/or activates an immune pathway not mediated by IL-18 in the bladder.

For example, the composition is formulated for intravesical administration to the bladder.

This disclosure relates to a method for selecting a subject for bladder cancer therapy, comprising: a) providing a sample from a subject with bladder cancer; b) determining from the sample the presence, absence, or level of a biomarker that correlates with interleukin 18 binding protein (IL-18 BP) expression by bladder cells from the subject; and wherein when the biomarker positively correlates with IL-18 BP expression and the biomarker is present or has a level at or above a first predetermined threshold, or when the biomarker negatively correlates with IL-18 BP expression and the biomarker is absent or has a level at or below a second predetermined threshold, the subject is identified as a candidate for a therapy selected from (a) (i) bacillus Calmette-Guérin (BCG) and (ii) at least one of interleukin 18 (IL-18), an inhibitor of IL-18 BP, an antagonist of an activator of IL-18 BP, and an agonist of a down-regulator of IL-18 BP or (b) a therapy that does not comprise BCG.

For example, the biomarker is IFN regulatory factor 1 (IRF1) and/or CCAAT/enhancer binding protein β (C/EBPB).

This disclosure relates to a method for determining treatment for bladder cancer, comprising providing bladder cells from a subject with bladder cancer; culturing a portion of the cells with IFNγ and separately culturing a portion of the cells with IFNγ and TNFα, each culture maintained for a period of time sufficient to induce expression of interleukin 18 binding protein (IL-18 BP); determining expression level of IL-18 BP and uroplakin 1B, by the cells cultured in b); determining the ratio of expression level of IL-18 BP to uroplakin 1b for cells cultured with IFNγ (IL-18BP:uroplakin 1b IFNγ) and for cells cultured with IFNγ and TNFα (IL-18BP:uroplakin 1b IFNγ+TNFα); determining the (IL-18BP:uroplakin 1b IFNγ):(IL-18BP:uroplakin 1b IFNγ+TNFα) ratio, wherein a (IL-18BP:uroplakin 1b IFNγ):(IL-18BP:uroplakin 1b IFNγ+TNFα) ratio of about 0.2 or greater indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy. The bladder cells preferably include urothelium and bladder cancer cells.

For example, the value of (IL-18BP:uroplakin 1b IFNγ):(IL-18BP:uroplakin 1b IFNγ+TNFα) indicating that the subject should receive therapy is about 0.2 or above, about 0.3 or above, about 0.4 or above, about 0.5 or above, about 0.6 or above, about 0.7 or above, about 0.8 or above, about 0.9 or above, about 1.0 or above, about 1.2 or above, about 1.4 or above, about 1.6 or above, about 1.8 or above, or about 2.0 or above.

For example, the culture period in b) is selected from at least about 12 hours, at least about 24 hours, at least about 36 hours, at least about 48 hours, at least about 60 hours, at least about 72 hours, at least about 84 hours, or at least about 96 hours.

For example, determining the expression level in c) comprises determining a protein level.

For example, determining the expression level in c) comprises determining a mRNA level.

For example, the inhibitor, the antagonist, or the agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP expression; agonists of a down-regulator of IL-18 BP transcription; a polynucleotide for gene therapy to reduce expression of IL-18 BP by non-cancerous urothelial cells, bladder cancer cells, or both: a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

For example, the inhibitor of IL-18BP is an anti-IL18BP antibody.

The method can further comprise administering the therapy to the subject with bladder cancer.

For example, the therapy is administered by intravesical administration to the bladder.

For example, the bladder is heated.

For example, the at least one of the inhibitor, antagonist, and agonist reduces binding of IL-18 BP to IL-18 in the bladder or reduces IL-18 BP levels in the bladder.

For example, the administering comprises intravesicular administration of an ASO complementary to an RNA encoding IL-18 BP isoform A (IL-18 BPa), to an RNA encoding IL-18 BP isoform C (IL-18 BPc), or to an RNA encoding the activator of IL-18 BP.

For example, the ASO comprises a sequence that, upon complementation of the RNA encoding IL-18 BP or the activator thereof by the ASO, causes at least one of reduces translation of the RNA and alters splicing of the RNA encoding IL-18 BP, thereby reducing the yield of IL-18 BPa and/or IL-18 BPc.

For example, bacillus Calmette-Guérin (BCG) is administered to the subject in need thereof; or wherein the subject in need thereof has received or will receive BCG. For example, the therapy comprises BCG and at least one of interleukin 18 (IL-18), an inhibitor of IL-18 BP, an antagonist of an activator of IL-18 BP, and an agonist of a down-regulator of IL-18 BP.

For example, the BCG is administered by intravesical administration to the bladder.

For example, the IL-18, the inhibitor, the antagonist, and/or the agonist is/are administered essentially simultaneously with the BCG.

For example, the BCG therapy is a primary BCG therapy or a maintenance BCG therapy.

For example, the subject is identified as being non-responsive to BCG monotherapy prior to administering the IL-18, the inhibitor, the antagonist, and/or the agonist.

For example, the inhibitor of IL-18 BP reduces the binding of IL-18 BP isoform A (IL-18 BPa) and/or IL-18 BP isoform C (IL-18 BPc) to IL-18 and/or reduces levels of IL-18 BPa and/or IL-18 BPc.

For example, the non-BCG therapy is selected from the group consisting of chemotherapy, radiation therapy, immunotherapy, cystectomy, and combinations thereof.

This disclosure relates to a composition, comprising: an effective amount of interleukin 18 (IL-18), an inhibitor of IL-18 binding protein (IL-18 BP), an antagonist of an activator of IL-18 BP, and/or an agonist of a down-regulator of IL-18 BP, and a pharmaceutically-acceptable excipient.

For example, the composition is formulated for intravesical administration to the bladder.

For example, the inhibitor, antagonist, or agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

For example, the composition comprises an effective amount of bacillus Calmette-Guérin (BCG).

This disclosure relates to a kit, comprising: instructions for performing the method of any one of the preceding claims, and a composition comprising an effective amount of at least one of interleukin 18 (IL-18), an inhibitor of IL-18 binding protein (IL-18 BP), an antagonist of an activator of IL-18 BP, and an agonist of a down-regulator of IL-18 BP, and a pharmaceutically-acceptable carrier.

For example, the inhibitor reduces binding of IL-18 BP to IL-18 in the bladder, reduces IL-18 BP levels in the bladder, and/or activates an immune pathway not mediated by IL-18 in the bladder.

For example, the composition is formulated for intravesical administration to the bladder.

This disclosure relates to a method for selecting a subject for bladder cancer therapy, comprising: providing a sample from a subject with bladder cancer; determining from the sample the presence, absence, or level of a biomarker that correlates with interleukin 18 binding protein (IL-18 BP) expression by bladder cells from the subject; and when the biomarker positively correlates with IL-18 BP expression and the biomarker is present or has a level at or above a first predetermined threshold, or when the biomarker negatively correlates with IL-18 BP expression and the biomarker is absent or has a level at or below a second predetermined threshold, the subject is identified as a candidate for a therapy selected from (a) (i) bacillus Calmette-Guérin (BCG) and (ii) at least one of interleukin 18 (IL-18), an inhibitor of IL-18 BP, an antagonist of an activator of IL-18 BP, and an agonist of a down-regulator of IL-18 BP or (b) a therapy that does not comprise BCG.

For example, the biomarker is IFN regulatory factor 1 (IRF1) and/or CCAAT/enhancer binding protein β (C/EBPB).

This disclosure relates to a method for selecting a subject for bladder cancer therapy, comprising providing a sample from a subject with bladder cancer; determining from the sample the presence, absence, or level of a biomarker that correlates with interleukin 18 binding protein (IL-18 BP) expression by bladder cells from the subject; and when the biomarker positively correlates with IL-18 BP expression and the biomarker is present or has a level at or above a first predetermined threshold, or when the biomarker negatively correlates with IL-18 BP expression and the biomarker is absent or has a level at or below a second predetermined threshold, the subject is identified as a candidate for a therapy selected from (a) (i) bacillus Calmette-Guérin (BCG) and (ii) interleukin 18 (IL-18), an inhibitor of IL-18 BP, an antagonist of an activator of IL-18 BP, and/or an agonist of a down-regulator of IL-18 BP or (b) a therapy that does not comprise BCG.

The biomarker may be genetic marker, or a protein level and/or an RNA level of IL-18 BP or of a polypeptide that is correlated with or indicative of IL-18 BP expression. In embodiments, the biomarker is IFN regulatory factor 1 (IRF1) and/or CCAAT/enhancer binding protein β (C/EBPβ).

In embodiments, the genetic marker may be a single nucleotide polymorphism (SNP), such as an SNP within or near the IL-18 BP transcriptional promoter.

The method may further comprise identifying the subject as a candidate for a therapy consisting essentially of BCG, when the biomarker positively correlates with IL-18 BP expression and the biomarker is absent or has a level at or below the first predetermined threshold, or when the biomarker negatively correlates with IL-18 BP expression and the biomarker is present or has a level at or above the second predetermined threshold.

Determining the presence, absence, or level of the biomarker may comprise any appropriate technique. For example, determining the protein and/or the RNA level of IL-18 BP or a polypeptide biomarker may comprise isolating urothelial cells from the sample, wherein the sample is a bladder biopsy; and determining the protein and/or the RNA level of interleukin 18 binding protein (IL-18 BP) or the biomarker produced by the isolated cells.

The method may also comprise normalizing the protein and/or the RNA level of IL-18 BP or the biomarker. For example, normalizing may be to the protein and/or RNA level of at least one gene (i) selective for urothelium and not bladder muscle and (ii) not regulated by IFNγ, such as a uroplakin, e.g. uroplakin 2B or uroplakin 1A.

The method may further comprise exposing urothelial cells of the sample to IFNγ prior to determining the presence, absence, or level of the biomarker: such as by providing the urothelial cells with a medium comprising from 0.1 ng/mL IFNγ to 500 ng/ml IFNγ; and/or exposing for about 48 hours.

In the method, determining the protein and/or the RNA level of IL-18 BP may comprise quantifying the protein and/or the RNA level of IL-18 BP isoforms.

In the method, determining the level of the IL-18 BP may comprise performing reverse-transcriptase quantitative PCR (RT-qPCR) on RNA encoding IL-18 BP; and/or performing an enzyme-linked immunosorbent assay (ELISA) on IL-18 BP, among other techniques.

The first threshold value may be from 5 μg/mL IL-18 BP secreted in up to 2 mL of culture medium per 100,000 cells after 48 hours of exposure to 1 ng/ml or more of IFNγ to 1000 μg/mL secreted in up to 2 mL of culture medium IL-18 BP per 100,000 cells after 48 hours of exposure to 1 ng/ml or more of IFNγ, such as a threshold value of 20 μg/mL IL-18 BP secreted in up to 2 mL of culture medium per 100,000 cells after 48 hours of exposure to 1 ng/ml or more of IFNγ.

The therapy that does not comprise BCG may be selected from the group consisting of chemotherapy, radiation therapy, immunotherapy, cystectomy, and combinations thereof: such as chemotherapy.

The disclosure also relates to a kit, comprising instructions for performing a method described herein, and RT-qPCR primers for the amplification of RNA encoding IL-18 BP and/or an ELISA testing unit comprising an antibody against IL-18 BP protein of the species of which the subject is a member, e.g., an antibody specific for human IL-18 BPa.

This disclosure relates to a method for treating bladder cancer, comprising administering to a subject in need thereof an effective amount of interleukin 18 (IL-18), an inhibitor of IL-18 binding protein (IL-18 BP), an antagonist of an activator of IL-18 BP, and/or an agonist of a down-regulator of IL-18 BP. In an example, treatment include administration of BCG, interleukin 18 (IL-18), an inhibitor of IL-18 binding protein (IL-18 BP), an antagonist of an activator of IL-18 BP, and/or an agonist of a down-regulator of IL-18 BP.

The inhibitor, antagonist, or agonist may reduce binding of IL-18 BP to IL-18 in the bladder or reduce IL-18 BP levels in the bladder.

The IL-18 or the inhibitor, antagonist, or agonist may be administered by intravesical administration to the bladder.

The inhibitor, antagonist, or agonist may be selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP expression; and combinations thereof.

The method may further comprise heating the bladder.

The method may further comprise administering bacillus Calmette-Guérin (BCG) to the subject in need thereof; or wherein the subject in need thereof has received or will receive BCG. The BCG may be administered by intravesical administration to the bladder. The IL-18, the inhibitor, the antagonist, and/or the agonist may be administered essentially simultaneously with the BCG. The BCG therapy may be a primary BCG therapy or a maintenance BCG therapy.

The method may further comprise identifying the subject as being non-responsive to BCG monotherapy prior to administering the IL-18, the inhibitor, the antagonist, and/or the agonist.

The administering may comprise intravesicular administration of an ASO complementary to an RNA encoding IL-18 BP isoform A (IL-18 BPa) and/or isoform C (IL-18 BPc) or to an RNA encoding the activator of IL-18 BP. The ASO may comprise a sequence that, upon complementation of the RNA encoding IL-18 BP or the activator thereof by the ASO, reduces translation of the RNA, modifies splicing of the RNA encoding IL-18 BP to yield less bioactive IL-18 BP (e.g., less IL-18 BPa and/or less IL-18 BPc), or both.

The inhibitor, antagonist, or agonist may reduce the binding of IL-18 BP to IL-18 and/or reduce levels of IL-18 BP. For example, it may reduce the binding and/or the levels of IL-18 BPa and/or IL-18 BPc.

After administering IL-18, the inhibitor, the antagonist, and/or the agonist (and BCG, if BCG is administered), the method may further comprise administering a third therapy other than IL-18, the inhibitor, the antagonist, and/or the agonist (and BCG, if BCG is administered). For example, the third therapy may be selected from the group consisting of chemotherapy, radiation therapy, non-BCG immunotherapy, cystectomy, and combinations thereof. For a particular example, the third therapy may be chemotherapy.

This disclosure also relates to a composition, comprising an effective amount of interleukin 18 (IL-18), the inhibitor, the antagonist, and/or the agonist, and a pharmaceutically-acceptable excipient. The composition may be formulated for intravesical administration to the bladder.

In the composition, the inhibitor, the antagonist, and/or the agonist may be selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both: a decoy polynucleotide for a regulatory sequence that promotes or enhances IL-18 BP expression; and combinations thereof.

In the composition, the inhibitor and/or the antagonist may be an ASO complementary to an RNA encoding IL-18 BP isoform A (IL-18 BPa) and/or isoform C (IL-18 BPc) or to an RNA encoding the activator of IL-18 BP.

An ASO may comprise a sequence such that complementation of the RNA encoding IL-18 BP or the activator thereof by the ASO reduces translation of the RNA, reduces splicing of the RNA encoding IL-18 BP to yield less bioactive IL-18 BP, or both.

The composition may further comprise an effective amount of bacillus Calmette-Guérin (BCG).

This disclosure also relates to a kit, comprising instructions for performing a method described herein, and a composition comprising an effective amount of interleukin 18 (IL-18), the inhibitor, the antagonist, and/or the agonist, and a pharmaceutically-acceptable carrier. Particular examples of the composition are described herein.

This disclosure also relates to a method for determining treatment for bladder cancer, comprising providing bladder cells from a subject with bladder cancer: culturing at least a portion of the cells with IFNγ and/or culturing at least a portion of the cells with IFNγ and TNFα, each culture maintained for a period of time sufficient to induce expression of interleukin 18 binding protein (IL-18 BP) and/or IL-18 by the cells; determining expression level of IL-18 BP and a gene product selected from the group consisting of i) a gene product that is expressed in urothelium and preferably not in other bladder cells, ii) a gene product for which the levels do not change when bladder cells are exposed to IFNγ, and iii) a gene product that is decreased in bladder cancer patients who do not respond to BCG therapy or who experience recurrence following BCG therapy by the cells cultured in b); and determining the ratio of expression level of IL-18 BP to the gene product wherein a ratio indicative of a level of IL-18BP that is expected to interfere with BCG or IFNγ therapy indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy.

### 4. BRIEF DESCRIPTION OF THE DRAWINGS

FIG. 1 illustrates the state of the art regarding BCG therapy for bladder cancer, depicting immune system responses and possible routes of inhibition thereof, prior to the reporting of information presented herein.

FIG. 2a shows IL-18 BPa protein is induced in six bladder cancer cell lines by IFNγ, as described in Example 1.

FIG. 2b shows that IL-18 BPa mRNA is induced in six bladder cancer cell lines by IFNγ, as described in Example 1.

FIG. 3 shows IL-18 BPa protein production is induced in normal bladder urothelial cells by IFNγ, as described in Example 2.

FIG. 4 shows that conditioned media from bladder cancer cell lines exposed to IFNγ has IL-18 neutralizing activity that can be partially blocked by an anti-IL-18BPa specific antibody.

FIG. 5 shows that IL-18 BPa mRNA is induced in six bladder cancer cell lines by IFNγ, as described in Example 1.

FIG. 6 shows that IL-18 BPa protein is induced in six bladder cancer cell lines by IFNγ, as described in Example 1.

FIG. 7 shows that IL-18 BPa mRNA is induced by IFNγ in fresh normal-appearing bladder biopsy from a patient with bladder cancer, as described in Example 2.

FIG. 8 shows the effect of medium on IL-18 BPa mRNA induction by IFNγ in fresh normal-appearing bladder biopsy from a patient with bladder cancer, as described in Example 2.

FIG. 9 shows that IL-18 BPa mRNA may be induced by IFNγ and TNF in fresh normal-appearing bladder biopsy from a patient with bladder cancer, as described in Example 2.

FIG. 10 shows relative expression of IL-18BPa in conditioned media.

FIG. 11A-11C shows IL-18BPa mRNA change relative to Uroplakin 1B on exposure of normal urothelium to IFNγ.

FIG. 12A-12C shows IL-18BPa mRNA change relative to Uroplakin 1B on exposure of normal urothelium to the combination of IFNγ and TNFα.

FIG. 13A-13C shows IL-18BPa mRNA change relative to Uroplakin 1B on exposure of normal urothelium to the ratio of IFNγ/IFNγ+TNFα.

FIG. 14 shows that IL-18BPa expression was induced by IFNγ and that siRNAs directed against IL-18BPa inhibited the IL-18BPa expression.

FIG. 15 shows IL-18BPa concentration in the medium upon exposure to IRF-1 or STAT1 transcription factor decoys and that the IRF-1 decoy inhibited IFNγ induced expression of IL-18BPa.

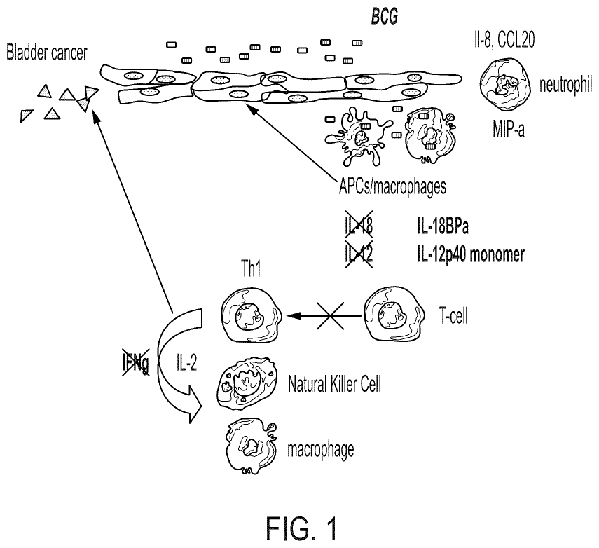

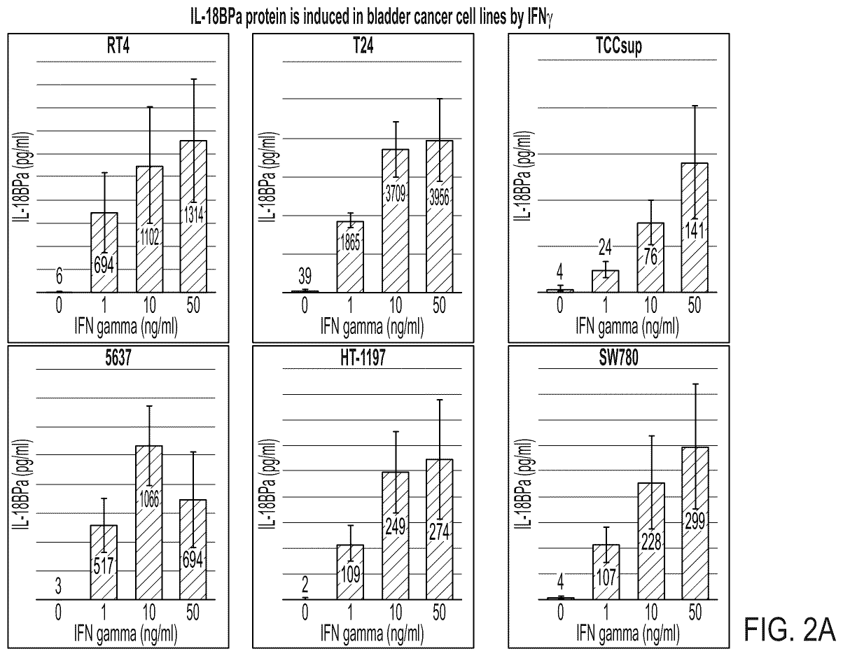

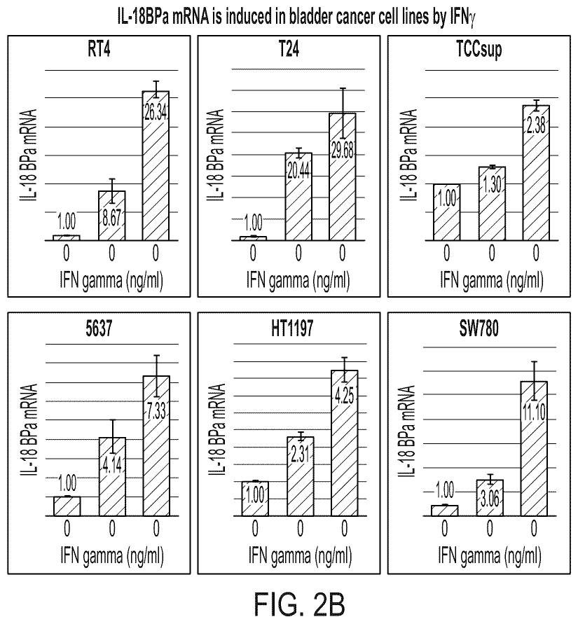

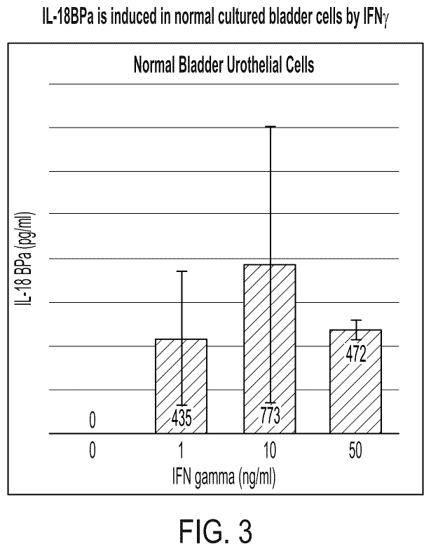

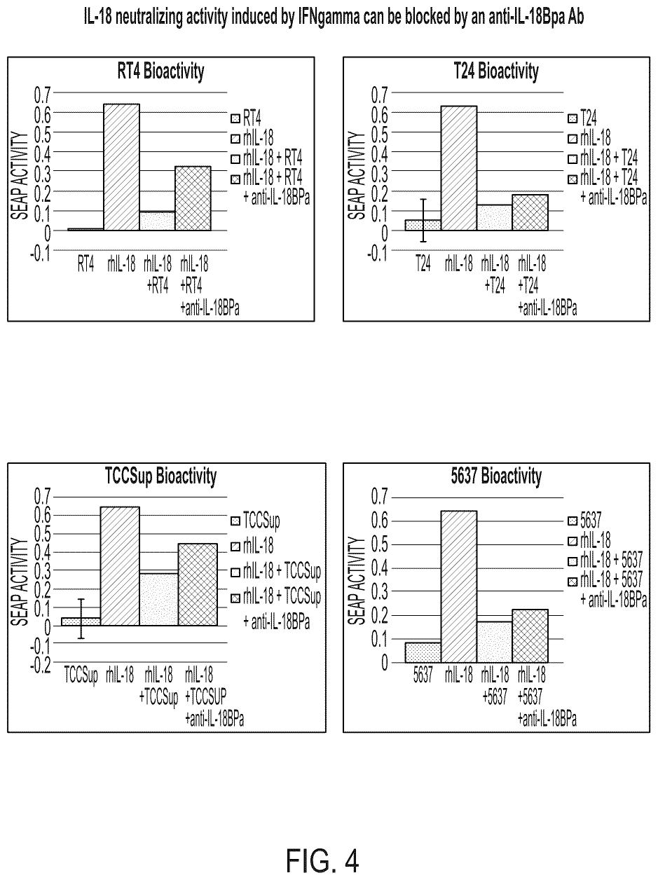

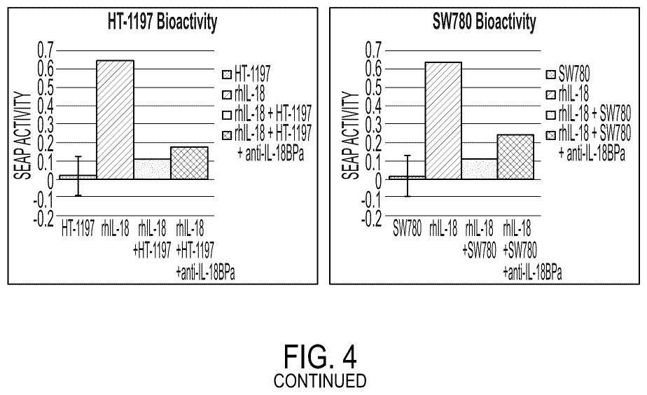

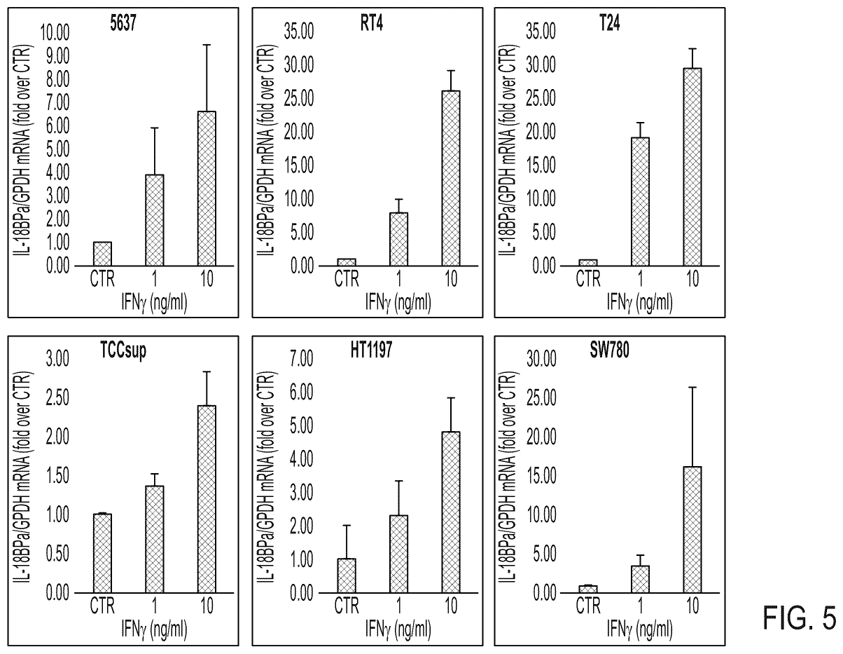

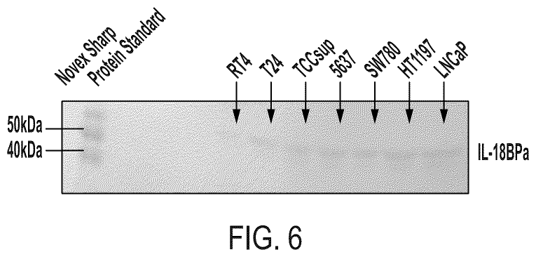

### 5. DETAILED DESCRIPTION

This disclosure relates to the stratification of subjects suffering from non-invasive bladder cancer into those expected to respond to BCG therapy and those not expected to respond. The expected response or lack thereof can be predicted from the presence, absence, or level of various biomarkers, such as from in vitro determination of levels of interleukin 18 binding protein (IL-18 BP), e.g. IL-18 BP isoform A (IL-18 BPa), for example, IL-18 BP levels induced by interferon gamma (IFNγ) in the subject's urothelial cells, including in the subject's non-cancerous urothelial cells. This disclosure also relates to the treatment of subjects suffering from non-invasive bladder cancer who would be expected to not respond to BCG therapy under the current standard of care. The treatment involves BCG therapy in combination with IL-18 and/or an inhibitor of IL-18 BP that reduces binding of an interleukin-18 binding protein (IL-18 BP) to interleukin-18 (IL-18) in the subject's bladder.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include.” “such as,” and the like are intended to convey inclusion without limitation, unless otherwise specifically indicated.

Unless otherwise indicated, the terms “at least.” “less than,” and “about.” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

The term “biopsy,” when used as a verb, refers to the extraction of tissue, which may comprise healthy cells, diseased cells, or a mixture thereof, from a living body of a subject. When used as a noun, it refers to tissue extracted from a living body of a subject.

The terms “level produced.” “produced level,” and cognates thereof refer to amounts of a material present in a cell, secreted into a medium, or the like at a particular timepoint after an event expected to trigger production of the material by cells of interest. This term is not limited to the gross amount of the material produced by the cell, but instead refers to the net amount remaining after the operation of cellular and/or extracellular processes that may lower the amount of the material.

The term “genetic marker” refers to one or more single or multiple nucleotide polymorphisms or other DNA sequence variations or gene expression patterns. The genetic marker may be a sequence variation in germline DNA (i.e., in DNA inherited from the subject's parents), a somatic mutation (i.e., a tumor-acquired change in DNA), or a combination thereof.

The term “in the bladder” as used herein refers to both cells of the bladder wall and the volume contained within the bladder wall.

### Immune Responses in Bladder Cancer and Non-Cancerous Urothelial Cells

IL-18 is an interferon gamma (IFNγ) stimulatory factor. Mice lacking IL-18 have a reduced natural killer cell response and markedly reduced IFNγ production. (Takeda et al., 1998) IL-18 BP binds IL-18 and inhibits its ability to bind the IL-18 receptor and consequently stimulate the production of IFNγ. (Dinarello. 2000)

Intravesical BCG immunotherapy is the standard of care for superficial bladder cancer. Despite its widespread use, approximately 30% and up to about 50% of patients do not respond to BCG immunotherapy, and must progress to other therapeutic approaches. (Herr et al., 2008) BCG efficacy has been shown to correlate with, and be dependent on, expression of IFNγ in the bladder. (Luo et al. 2013) Also, cancer recurrence in patients that have received BCG immunotherapy is associated with higher urinary levels of IL-18 BPa when comparing baseline to week 13 post initiation of intravesical BCG therapy (Salmasi et al. 2019) and lower IL-18 production during the first 6 or 12 hrs after BCG therapy. (Thalmann et al. 2000).

Without wishing to be bound by any particular theory or mechanism, it is believed that bladder cancer cells might produce increased amounts of IL-18 BP as a defense against the anti-cancer immune responses (e.g., increased production of IFNγ) induced by BCG. Our work in Example 1 agrees with this possibility.

In addition, the studies reported herein demonstrate for the first time, that exposure of non-cancerous urothelial cells to IFNγ induces expression of IL-18 BP, which binds to and inhibits the biological activity of IL-18 including IL-18 induced production of IFNγ. See Example 2 for more detail. To reiterate, the studies exemplified herein demonstrate that the IFNγ-induced increase in IL-18 BP levels occurs in non-cancerous urothelial cells and in bladder cancer cells.

Though IL-18 BP expression normally serves a desirable function, namely, to reversibly bind to and thus inhibit IL-18 binding to IL-18 receptor, thereby preventing excessive immune responses (e.g., cytokine storms). IL-18 BP expression is undesirable in the context of therapy for bladder cancer. Deactivation of IL-18 during BCG therapy inhibits the recruitment of neutrophils, antigen-presenting cells (APCs), macrophages, helper T cells, and/or natural killer cells to further attack both BCG and bladder cancer cells. As a result, our studies demonstrate the strength of induction of expression of IL-18 BP by IFNγ in bladder cells would be inversely related to the efficacy of BCG treatment for bladder cancer. In other words, the stronger the induction of expression of IL-18 BP by IFNγ in cells of a subject's bladder, the poorer the expected efficacy of BCG treatment for that subject, and vice versa.

The current disclosure relates to the novel and unexpected finding that not just human bladder cancer cells, but also normal mammalian urothelial cells, produce bioactive IL-18 BP when exposed to IFNγ. Neither bladder cancer cells nor non-cancerous urothelial cells produce IL-18 BP under IFNγ-free culture conditions. In addition, studies reported herein show that the level of IL-18 BP that normal mammalian urothelial cells and human bladder cancer cells produce in vitro is positively correlated with the concentration of IFNγ in the medium (e.g., there is a dose response relationship). Without wishing to be bound by any particular theory or mechanism, it is believed that production of IL-18 BP by bladder cancer and normal urothelium cells upon exposure to IFNγ functions as a functional feedback inhibition system that inhibits the Th1 immune response.

Accordingly, production by both normal urothelium and bladder cancer cells of IL-18 BP on exposure to IFNγ may reduce the efficacy of intravesical BCG therapy for bladder cancer. Inhibition of the urothelial and bladder cancer cell derived IL-18 BP activity represents a novel method for treating bladder cancer and of improving response to BCG in patients with superficial bladder cancer. Furthermore, determining the amount of IL-18 BP expressed by normal urothelium, in response to exposure to IFNγ, may be a useful tool in predicting BCG response.

### Identification of BCG Responders and Non-Responders

In view of our observations that the amount of IL-18 BP, such as IL-18 BPa, expressed by normal urothelium in response to exposure to IFNγ may be a useful tool in predicting BCG response, subjects may be identified as BCG responders (“being responsive to BCG therapy”) or BCG non-responders (“being non-responsive to BCG therapy”) based on one or more aspects of the relationship between IFNγ and IL-18 BP expression.

For example, a subject may be identified as a responder or a non-responder based on one or more biomarkers, i.e., genetic and/or phenotypic markers of IL-18 BP expression. For example, a patient may have one or more genomic sequences regulating the rate of transcription of sequences encoding IL-18 BP, transcript splicing to yield IL-18 BP isoforms, the rate of translation of an IL-18 BP polypeptide, or two or more thereof. If such sequences in the subject increase the transcription rate of IL-18 BP, increase the fraction of transcripts yielding the IL-18 BPa isoform, increase the translation of an IL-18 BP polypeptide, or two or more thereof relative to the baseline rate or fraction of all subjects or subjects known to respond to BCG therapy, the subject may be identified as a BCG non-responder. Sequences that reduce the transcription rate or decrease the fraction of transcripts yielding an IL-18 BP, e.g., IL-18 BPa, relative to baselines may allow the subject to be identified as a BCG responder.

For another example, various diseases or conditions may be associated with systemically increased or decreased IL-18 BP levels, which may correlate with IL-18 BP production by urothelial cells. For example, IL-18 BP levels are elevated in Crohn's disease. IL-18 BP levels may be decreased in one or more autoimmune disorders.

Transcription of the IL-18 BP gene has been shown to be regulated by Interferon regulatory factor-1 (IRF-1). (https://www.pnas.org/doi/10.1073/pnas.262663399) Single nucleotide polymorphisms (SNPs) in the IRF-1 gene and its regulatory sequence have been shown to affect the expression of IRF-1. (https://www.atsjournals.org/doi/10.1164/rccm.200703-3730C) SNPs have also been found in IRF-1 DNA binding sites that affect the transcription of IRF-1 regulated genes. (IL-18 promoter polymorphisms have been shown to affect inducibility of the IL-18 promoter and to correlate with various disease states. (https://pubmed.ncbi.nlm.nih.gov/11108943/) (https://www.nature.com/articles/6364183)

The present disclosure relates in part to using the relationship between IFNγ and IL-18 BP expression to select a subject for bladder cancer therapy, select a bladder cancer therapy for a subject, optimize a bladder cancer therapy, and/or select a subject for conditioning or pre-conditioning of BCG therapy. This selection/optimization may include one or more of providing a sample from a subject with bladder cancer; determining from the sample the presence, absence, or level of a biomarker that correlates with interleukin 18 binding protein (IL-18 BP) expression by bladder cells from the subject; and when the biomarker positively correlates with IL-18 BP expression and the biomarker is present or has a level at or above a first predetermined threshold, or when the biomarker negatively correlates with IL-18 BP expression and the biomarker is absent or has a level at or below a second predetermined threshold, the subject is identified as a candidate for a therapy selected from (a) (i) bacillus Calmette-Guérin (BCG) and (ii) interleukin 18 (IL-18), an inhibitor of IL-18 BP, an antagonist of an activator of IL-18 BP, and/or an agonist of a down-regulator of IL-18 BP or (b) a therapy that does not comprise BCG. For example, the threshold value may be 20 μg/mL IL-18 BP per 100,000 cells after 48 hours of exposure to 1 ng/ml or more of IFNγ. Related to this selection/optimization, the subject may be administered a therapy consisting essentially of BCG, if the if the biomarker is present at less than about the threshold value.

Alternatively or in addition, an aspect of the relationship between IFNγ and IL-18 BP expression that can be used to determine or predict an IL-18 BP level. (which may be as part of a process to identify a subject as a BCG responder or a BCG non-responder) is quantifying IL-18 BP expression by bladder cells, including bladder cancer cells and non-cancerous urothelial cells, when exposed to IFNγ. The exposure may be in vitro or in vivo. An in vitro example will be described in more detail herein.

For example, a method for determining treatment for bladder cancer can utilize culturing at least a portion of bladder cells from a subject with bladder cancer with IFNγ and/or culturing at least a portion of the cells with IFNγ and TNFα, and maintaining each culture maintained for a period of time sufficient to induce expression of interleukin 18 binding protein (IL-18 BP) and/or IL-18 by the cells. The culture can be maintained, for example, for at least about 12 hours, at least about 24 hours, at least about 36 hours, at least about 48 hours, at least about 60) hours, at least about 72 hours, at least about 84 hours, or at least about 96 hours. The expression levels in the culture are determined for IL-18 BP and a gene product selected from the group consisting of i) a gene product that is expressed in urothelium and preferably not in other bladder cells, ii) a gene product for which the levels do not change when bladder cells are exposed to IFNγ, and iii) a gene product that is decreased in bladder cancer patients who do not respond to BCG therapy or who experience recurrence following BCG therapy. Additionally, the ratio of expression level of IL-18 BP to the gene product is determined wherein a ratio indicative of a level of IL-18BP that is expected to interfere with BCG or IFNγ therapy indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy.

Suitable gene products that are expressed in urothelium and preferably not in other bladder cells are known to the skilled artisan and include, for example, mRNA an proteins encoded by the uroplakin 1b (Hs.01041715_m1) gene.

Suitable gene products for which the levels do not change appreciably when bladder cells are exposed to IFNγ are known to the skilled artisan and include, for example, mRNA and proteins encoded by the PLA2G2F (urothelium specific). SLC15A3 (not exclusive to urothelium). ATF3 (not exclusive to urothelium). DDX60) (not exclusive to urothelium). OASI (not exclusive to urothelium), and SP110 (not exclusive to urothelium) genes, and housekeeping genes such as GAPDH (Hs.02786624_g1) and ribosomal protein S23 (Hs01922548_s1).

Suitable gene products that are decreased in bladder cancer patients who do not respond to BCG therapy or who experience recurrence following BCG therapy are known to the skilled artisan. (See, e.g. Kim et al., 2010) Gene products that are decreased in patient who experience reoccurrence follow BCG therapy include, for example, mRNA and protein encoded by the MAEA (Hs.139896), SEC24C (Hs.81964), ZC3HC1 (Hs.194157), RBAF600 (Hs.148078), AP1G1 (Hs.461253), UBE21 (Hs.302903), HLA-A (Hs.181244), RNPS1 (Hs.355643), PTPRM (Hs.49774), BTG2 (Hs.519162), and PITRM1 (Hs.528300) genes. Gene products that are decreased in patients who experience progression following BCG therapy include, for example, mRNA and proteins encoded by the POLR2A (Hs.270017), STOML2 (Hs.3439), RNF20 (Hs.656088), XRCC2 (Hs.388739), SEPX1 (Hs.655346), TAX1BP3 (Hs. 12956), and LGMN (Hs.719135) genes. ( ).

A ratio of IL-18 BP to the gene product indicative of a level of IL-18BP that is expected to interfere with BCG or IFNγ therapy indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy. For example, therapy can include BCG and at least one of interleukin 18 (IL-18), an inhibitor of IL-18 BP, an antagonist of an activator of IL-18 BP, and an agonist of a down-regulator of IL-18 BP.

As described and exemplified herein, a ratio of IL-18 BP to the gene product can indicate that IL-18 BP will be induced by treatment to a level that inhibits immune activation and destruction of bladder cancer cells. For example as described in Table 3, a ratio of IL-18 BP to uroplakin upon exposure to IFNγ of 6.0 or higher is indicative that the subject should receive therapy that includes at least one of interleukin 18 (IL-18), an inhibitor of IL-18 BP, an antagonist of an activator of IL-18 BP, and an agonist of a down-regulator of IL-18 BP or a non-BCG therapy. In various embodiments, the ratio for IL-18BP:uroplakin 1b IFNγ indicating a need for therapy that includes at least one of interleukin 18 (IL-18), an inhibitor of IL-18 BP, an antagonist of an activator of IL-18 BP, and an agonist of a down-regulator of IL-18 BP or a non-BCG therapy is about 6.0 or greater, about 8.0 or greater, about 10.0 or greater, about 12.0 or greater, about 14.0 or greater, about 16.0 or greater, about 18.0 or greater, about 20.0 or greater, or about 22.0 or greater. It is expected that ratios of IL-18 BP to other gene products as detailed above, rather than uroplakin 1b, would be comparable.

In another example, as described and exemplified in Table 3, a ratio of IL-18 BP to uroplakin upon exposure to IFNγ+TNFα of 40 or lower is indicative that the subject should receive therapy that includes at least one of interleukin 18 (IL-18), an inhibitor of IL-18 BP, an antagonist of an activator of IL-18 BP, and an agonist of a down-regulator of IL-18 BP or a non-BCG therapy.

In various embodiments, the ratio for IL-18BP:uroplakin 1b IFNγ+TNFα indicating a need for therapy that includes at least one of interleukin 18 (IL-18), an inhibitor of IL-18 BP, an antagonist of an activator of IL-18 BP, and an agonist of a down-regulator of IL-18 BP or a non-BCG therapy is about 40 or lower, about 36 or lower, about 32 or lower, about 28 or lower, about 24 or lower, about 20 or lower, about 16 or lower, about 12 or lower, about 8 or lower, or about 4 or lower. It is expected that ratios of IL-18 BP to other gene products as detailed above, rather than uroplakin 1b, would be comparable.

As described and exemplified herein, it is preferred to determine a ratio of ratios. In particular, the ratio of IL-18 BP to uroplakin 1b when cells are cultured in IFNγ (the IL-18BP:uroplankin 1b IFNγ) and when cells are cultured in IFNγ+TNF alpha (the IL-18BP:uroplankin 1b IFNγ+TNF alpha). For example, as described in Table 3, a ratio of (IL-18BP:uroplankin 1b IFNγ):(the IL-18BP:uroplankin 1b IFNγ+TNF alpha) is then determined, and a ratio of 0.2 or higher is indicative that the subject should receive therapy that includes at least one of interleukin 18 (IL-18), an inhibitor of IL-18 BP, an antagonist of an activator of IL-18 BP, and an agonist of a down-regulator of IL-18 BP or a non-BCG therapy. In various embodiments, the ratio of (IL-18BP:uroplakin 1b IFNγ):(IL-18BP:uroplakin 1b IFNγ+TNFα) indicating that the subject should receive therapy that includes at least one of interleukin 18 (IL-18), an inhibitor of IL-18 BP, an antagonist of an activator of IL-18 BP, and an agonist of a down-regulator of IL-18 BP or a non-BCG therapy is about 0.2 or above, about 0.3 or above, about 0.4 or above, about 0.5 or above, about 0.6 or above, about 0.7 or above, about 0.8 or above, about 0.9 or above, about 1.0 or above, about 1.2 or above, about 1.4 or above, about 1.6 or above, about 1.8 or above, or about 2.0 or above. It is expected that ratios using other gene products detailed above rather than uroplakin 1b would be comparable.

The expression of uroplakin 1b decreases in bladder cancer cells compared to non-cancerous bladder cells. The expression of IL-18BP increases in subjects in which bladder cancer will recures. By having a method of prediction in which IL-18BP is in the numerator and uroplakin1b (or other suitable gene product) is in the denominator, it allows for powerful indicator of a negative prognosis and provides for a method of selection of a subject in need of the therapy disclosed herein.

### Biomarkers

Any suitable biomarker can be used according to this disclosure, including, e.g., genetic markers, a protein level and/or an RNA level of IL-18 BP or a polypeptide that is correlated with or indicative of IL-18 BP expression. Of particular interest is IL-18 BP expression in urothelium and/or bladder cancer cells.

When genetic markers are used as biomarkers, they may be found in nucleic acids (e.g., DNA or RNA) from bladder tumor cells or from healthy cells. Healthy cells can be collected from any of the subject's tissues. For example, buccal cells (from the lining of the oral cavity) can be collected using well-established and non-invasive techniques.

Biomarkers from nucleic acids (e.g., DNA, RNA) in bladder tumor cells and normal bladder cells can be collected as part of a bladder sample obtained, e.g., by biopsy or other suitable method. Determining IL-18 BP expression in urothelium and/or bladder cancer cells can be performed on cells collected as part of a bladder biopsy. Bladder biopsy is described below.

The biomarker can be positively correlated with IL-18 BP expression or negatively correlated with IL-18 BP expression.

### Isolation of Urothelial Cells

Urothelial cells form the interior lining of the bladder, ureters, and urethra. The present standard of care for most types of non-invasive bladder cancer involves performing a transurethral biopsy and resection of a tumor prior to subsequent treatment. Resection and/or biopsy generally removes a margin of non-cancerous urothelial cells, and commonly also bladder muscle cells and fibroblasts, around the tumor or putative tumor. Either cancerous urothelial cells, non-cancerous urothelial cells, or both may be used in the methods of this disclosure, e.g., to assess a biomarker. The biomarker may be a genetic marker from bladder cancer cells and/or non-cancerous urothelial cells, or IL-18 BP or a biomarker regulating or correlated with IL-18 BP levels that is isolated from bladder cancer cells and/or non-cancerous urothelial cells. Surprisingly, production of IL-18 BP by non-cancerous urothelial cells may be predictive of BCG response.

In embodiments, urothelial cells may be isolated from the biopsy (e.g., a sample obtained by biopsy), without the need for an additional transurethral procedure. If desired, a transurethral biopsy with the sole intent of extracting urothelial cells may be performed, such as in circumstances where surgery is not appropriate or where cells removed as part of a previous surgical resection were not retained or are not viable after retrieval from storage, among others that will be apparent to the person of ordinary skill in the art having the benefit of the present disclosure.

Although transurethral biopsy of the bladder is relatively non-invasive, biopsy routes other than the urethra may be used as desired.

Alternatively or in addition, because urothelial cells are generally present in voided urine, urothelial cells (e.g., non-cancerous urothelial cells) may be isolated from a urine sample provided by the subject. Such isolation may be performed using any suitable methods, such as cell counting and cell sorting techniques, for example. A voided urine sample may thus be a sample suitable for use in the methods of the present disclosure.

Accordingly, in embodiments, a bladder biopsy of the subject is provided. For the avoidance of doubt, the term “bladder biopsy” encompasses both biopsies intended to remove some or all of a tumor (which may also be referred to as “tumor biopsies”), and biopsies intended to remove only non-cancerous tissue.

The bladder biopsy may be used immediately or may be stored under appropriate conditions for a period of hours, days, weeks, or longer, or indefinitely. Appropriate conditions for maintaining and preserving the bladder biopsy for immediate use or for any particular desired duration of storage will be known to the person of ordinary skill in the art.

Regardless of the route by which a sample of urothelial cells is taken from the subject, in embodiments wherein non-cancerous urothelial cells are desired for use in subsequent operations, it may be appropriate or desirable to verify the cells are non-cancerous. This can be done by visual inspection, cytological techniques, molecular biology techniques, or the like, or combinations thereof. Such techniques are known to the person of ordinary skill in the art and need not be described further. Related to this, urothelial cells may be further processed to separate non-cancerous urothelial cells from cancerous cells, e.g. by fluorescence-activated cell sorting (FACS) or other techniques well-known to the person of ordinary skill in the art. The non-cancerous urothelial cells and/or the cancerous cells may be used in the methods described herein, depending on the biomarker to be examined and the cell type(s) in which the biomarker is present.

After isolation from the subject's urinary tract (optionally, including separation of non-cancerous urothelial cells from cancerous cells), desired urothelial cells may be cultured using suitable methods, labware, media, ambient conditions (e.g. temperature, humidity, composition of gas headspace, and the like), and other techniques known in the art. The labware may be a standard multiwell plate known in the art to which the urothelial cells may adhere and proliferate and into which a medium may be added over the urothelial cells. Alternatively or in addition, the medium may comprise 10% v/v serum, such as fetal calf serum (FCS) or fetal bovine serum (FBS).

The isolated urothelial cells may be cultured for a period of time prior to further operations, such as optionally exposing them to IFNγ as will be described below. The duration of culturing prior to exposure may be at least 15 minutes, such as at least about 30 minutes, at least about 1 hour, at least about 2 hours, at least about 3 hours, at least about 4 hours, at least about 5 hours, at least about 6 hours, at least about 8 hours, at least about 9 hours, at least about 12 hours, at least about 18 hours, at least about 24 hours, at least about 36 hours, at least about 48 hours, at least about 72 hours, or at least about 96 hours. The duration of culturing prior to exposure may be about 24 hours.

### Exposure of Urothelial Cells to IFNγ

After isolation, the urothelial cells (e.g., non-cancerous urothelial cells) may be exposed to IFNγ. Exposure may involve replacing the medium over the urothelial cells with the same or a similar medium comprising IFNγ. The concentration of IFNγ in the medium may be varied as desired. For example, the medium may comprise from about 0.1 ng/ml IFNγ to about 500 ng/mL IFNγ, such as from about 1 ng/mL IFNγ to about 50 ng/ml IFNγ.

In particular embodiments, the medium may comprise at least about 0.1 ng/ml IFNγ, at least about 0.2 ng/ml IFNγ, at least about 0.5 ng/ml IFNγ, at least about 1 ng/ml IFNγ, at least about 2 ng/ml IFNγ, at least about 5 ng/ml IFNγ, at least about 10 ng/ml IFNγ, at least about 15 ng/mL IFNγ, at least about 20 ng/ml IFNγ, at least about 25 ng/ml IFNγ, at least about 30 ng/ml IFNγ, at least about 35 ng/ml IFNγ, at least about 40 ng/ml IFNγ, at least 45 ng/ml IFNγ, or up to about 50 ng/ml IFNγ.

The duration of exposure may be at least 15 minutes, such as at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 12 hours, at least about 18 hours, at least about 24 hours, at least about 36 hours, at least about 48 hours, at least about 72 hours, or at least about 96 hours. For example, the duration of exposure may be about 48 hours. Desirably, the duration may be chosen to be sufficiently long for the urothelial cells to change their rate of expression of IL-18 BP in response to the exposure to IFNγ.

The exposure may be performed under the same or similar ambient conditions in which the isolated urothelial cells were cultured. Variations in the ambient conditions and other cell-culturing parameters of the exposure may be implemented by the person of ordinary skill in the art as a routine matter.

Presence, Absence, or Level of Biomarker Correlated with IL-18 BP Expression

Any technique for estimating, calculating, determining, or quantifying the presence, absence, or level of the biomarker may be used.

In embodiments, wherein the biomarker is a genetic marker, determining the presence or absence of the genetic marker, such as an SNP or a multi-nucleotide polymorphism, can be performed by techniques known to a person of ordinary skill in the art having the benefit of the present disclosure, such as single cell sequencing, FISH, PCR, using nucleic acid arrays and other well-known methods. The genetic marker may be determined from a genomic DNA sequence of the subject, without the need for a bladder biopsy, although DNA from cells provided by a bladder biopsy may be assayed for the presence or absence of the genetic marker.

As described herein, a number of genetic markers, such as single nucleotide polymorphisms (SNPs) and multi-nucleotide polymorphisms, are correlated with IL-18BP. Exemplary genetic markers include those described herein regarding interferon response factor 1 (IRF1). In embodiments, the genetic marker that correlates with IL-18 BP expression may be a SNP within or near the IL-18 BP transcriptional promoter.

The biomarker can be IL-18 BP expression or can be correlated with or indicative of IL-18 BP expression (or protein levels). Such biomarkers include, but are not necessarily limited to, transcription factors, nucleosomes, and DNA modifying enzymes. Such biomarkers may induce expression of IL-18 BP and/or may be protein or nucleic acid markers of IL-18BP expression and/or activity. Particular examples of such biomarkers include, but are not limited to, IFN regulatory factor 1 (IRF1) and CCAAT/enhancer binding protein β (C/EBPβ).

In embodiments, when the biomarker is IL-18 BP expression or is correlated with or indicative of IL-18 BP expression by urothelial cells, expression of IL-18 BP or a correlated/indicative polypeptide may be quantified based on the count of RNA transcripts, polypeptide molecules, or isoform molecules found in or secreted by the urothelial cells, such as after exposure to IFNγ.

Predicting, identifying, determining, etc. RNA levels may be of mRNA or other RNA molecules predictive of expression of IL-18 BP and/or a correlated/indicative polypeptide, such as micro RNA signatures.

Quantification of RNA can be performed by any of a number of techniques. In one particular technique, reverse-transcriptase PCR (RT-QPCR) may be performed to amplify RNA encoding IL-18 BP and/or a biomarker correlated with or indicative of IL-18 BP expression from urothelial cells, such as IFNγ-exposed urothelial cells and/or urothelial cells not exposed to IFNγ. For example, after a desired number of rounds of amplification, the ratio of amplicons from the IFNγ-exposed urothelial cells to amplicons from the non-exposed urothelial cells can be calculated. The ratio can be multiplied by a baseline concentration or amount of IL-18 BP protein expected to be produced by non-exposed urothelial cells to calculate a concentration or amount of IL-18 BP produced by the IFNγ-exposed urothelial cells.

Alternatively or in addition, quantification of RNA may involve normalizing the RNA level of IL-18 BP and/or the biomarker correlated with or indicative of IL-18 BP expression. Normalizing may allow comparison between samples that may differ in either or both of amount of tissue removed by biopsy and/or relative proportions of urothelium and non-urothelial tissue in the biopsy.

Normalizing may be to any known gene. For example, glycerol-3-phosphate dehydrogenase (GPDH) is commonly used as a normalizing gene in many applications. However, GPDH is present in all cells, and would thus be expected to be detectable in both urothelium and non-urothelial tissue.

Normalizing may be to the RNA expression level of at least one gene (i) selective for urothelium and not bladder muscle and (ii) not regulated by IFNγ. In other embodiments, for example, in embodiments wherein the urothelial cells are not exposed to IFNγ, the normalization gene may be selective for urothelium and not bladder muscle without regard to whether it is regulated by IFNγ. Housekeeping genes for urothelium are generally desirable candidates for a normalization gene.

By “selective for urothelium and not bladder muscle” is meant that the normalizing gene is not expressed at detectable levels in bladder muscle. An exemplary gene selective for urothelium and not bladder muscle is a uroplakin, such as the genes encoding uroplakin 2B or uroplakin 1A.

If a count of urothelial cells is available, normalizing may be to the urothelial cell count. This may be readily performed if the urothelial cells were isolated from voided urine.

Other techniques for amplification of nucleic acids can be used, for example, nucleic acid amplification can comprise polymerase chain reaction (PCR), reverse transcription PCR (RT-PCR), quantitative PCR (qPCR), reverse transcription qPCR (RT-qPCR), isothermal PCR, nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some embodiments the amplification is isothermal amplification. Isothermal nucleic acid amplification methods can therefore be carried out inside or outside of a laboratory environment. Examples of isothermal amplification methods include but are not limited to: loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

Protein quantification of IL-18 BP or a polypeptide biomarker correlated with or indicative of IL-18 BP expression may involve detection of the protein by any one of number of known techniques, such as western blotting (immunoblot), high-performance liquid chromatography (HPLC), liquid chromatography-mass spectrometry (LC/MS), antibody dependent methods such as enzyme-linked immunosorbent assay (ELISA), protein immunoprecipitation, protein immunostaining, protein chip techniques, or other protein detection techniques.

For example, IL-18 BP or the polypeptide biomarker may be quantified by performing ELISA using antibodies against IL-18 BP or the polypeptide of the species of which the subject is a member. ELISA-based quantification may be performed using a test unit similar to units for home or primary care physician (PCP) use for detection of viruses, testing for pregnancy, or the like.

Similarly to determination of RNA levels, determination of the protein level of IL-18 BP or the polypeptide biomarker may include comparison between a test sample and a control (e.g., protein levels produced after exposing urothelial cells to IFNγ vs. protein levels produced without IFNγ exposure) and/or normalizing the protein expression level of IL-18 BP or the polypeptide biomarker, such as to a uroplakin, such as uroplakin 2B or uroplakin 1A. Normalizing may alternatively or in addition include normalizing to urothelial cell count, if looking at cells in voided urine.

Regardless how the presence, absence, or level of the biomarker is determined, it may yield a qualitative or a numerical value of the amount or concentration that can be compared to a threshold value, as will be discussed below. The results of the comparison used to select a bladder cancer therapy.

As should be apparent, determination of the presence or absence of a biomarker can be quantitative or non-quantitative. For example, an assay can be used that detects the biomarker only when it is presence at or above a predetermined threshold level. Using such an assay, a positive test would indicate that the predetermined threshold value is met. Suitable assays and devices for such determinations are well-known and include, for example, lateral flow immune-assays, such as those used in home tests for PCP virus detection, pregnancy testing and the like.

Also, quantification techniques may have maximum values beyond which they cannot accurately quantify IL-18 BP or the biomarker. For example, in RT-PCR-based techniques, if the number of amplicons exceeds the number of primer molecules, each further round of amplification will fail to double the number of amplicons, and thus tend to underestimate the amount of mRNA encoding IL-18 BP or the biomarker in the original sample. For another example, in ELISA, if there are more IL-18 BP or biomarker molecules than anti-IL-18 BP or anti-biomarker antibody molecules, the excess IL-18 BP or biomarker molecules cannot be quantified. However, if the number of primer molecules in RT-PCR or the number of antibody molecules in ELISA are chosen to be sufficiently high, the threshold value can be set at or below the maximum accurate value from the technique, and the lack of accuracy will not impair the overall efficacy of the method.

### Threshold Values of Biomarker

As stated above, the presence, absence, or level of the biomarker in the sample can be compared to a first threshold value or a second threshold value. For example, the first threshold value may be chosen such that subjects with the biomarker absent (or present at a level below a predetermined threshold amount) may be expected to be successfully respond to bacillus Calmette-Guérin (BCG) therapy, whereas subjects with the biomarker present, such as at or above the first threshold value, may be expected to not respond to BCG monotherapy.

For another example, the second threshold value may be chosen such that subjects with the biomarker present, such as at a level above a predetermined threshold amount, may be expected to successfully respond to bacillus Calmette-Guérin (BCG) therapy, whereas subjects with the biomarker absent, or at or below the second threshold value, may be expected to not respond to BCG monotherapy.

The threshold may be any suitable value, depending on the biomarker that correlates with or is indicative of IL-18 BP expression or protein levels that inhibit IL-18 biological activity in the bladder (e.g., the urothelium).

In embodiments wherein the urothelial cells are exposed to IFNγ and the biomarker is IL-18 BP levels, the first threshold value may be at least 5 μg/mL IL-18 BP per 100.000 cells after 48 hours of exposure to 1 ng/ml or more of IFNγ, such as at least 10 μg/mL IL-18 BP, at least 20 μg/mL IL-18 BP, at least 50 μg/mL IL-18 BP, at least 100 μg/mL IL-18 BP, at least 200 μg/mL IL-18 BP, at least 300 μg/mL IL-18 BP, at least 400 μg/mL IL-18 BP, at least 500) pg/mL IL-18 BP, at least 600 μg/mL IL-18 BP, at least 700 μg/mL IL-18 BP, at least 800 μg/mL IL-18 BP, or at least 900 μg/mL IL-18 BP per 100.000 cells after 48 hours of IFNγ exposure.

For example, the first threshold value may have a value from 5 μg/mL IL-18 BP secreted in up to 2 mL of culture medium per 100.000 cells after 48 hours of exposure to 1 ng/ml or more of IFNγ to 1000 μg/mL IL-18 BP secreted in up to 2 mL of culture medium per 100.000 cells after 48 hours of exposure to 1 ng/ml or more of IFNγ.

For example, the first threshold value may be 20 μg/mL IL-18 BP secreted in up to 2 mL of culture medium per 100.000 cells after 48 hours of exposure to 1 ng/ml or more of IFNγ.

### Selection of Bladder Cancer Therapy in View of Biomarker Presence, Absence, or Level

The presence, absence, or level of a biomarker positively correlated with IL-18 BP expression will either be about the first threshold value or higher, or will be lower than about the first threshold value. The presence, absence, or level of a biomarker negatively correlated with IL-18 BP expression will either be about the second threshold value or lower, or will be higher than about the second threshold value.

If at about the first threshold value or higher, the positively-correlated biomarker suggests that the level of IL-18 BP may be sufficiently high that the subject would be expected not to respond to BCG monotherapy. Accordingly, to the subject may be administered a therapy selected from (a) (i) BCG and (ii) interleukin 18 (IL-18), an inhibitor of IL-18 BP, an antagonist of an activator of IL-18 BP, and/or an agonist of a down-regulator of IL-18 BP, or (b) a therapy that does not comprise BCG.

If the positively-correlated biomarker is absent, or present at a level below the first threshold value, the biomarker suggests that the level of IL-18 BP may be sufficiently low that the subject would be expected to respond to BCG monotherapy. Accordingly, to the subject may be administered a therapy consisting essentially of BCG. Alternatively, to the subject may be administered a therapy comprising BCG and at least one other therapy. For example, the at least one other therapy may include IL-18, an inhibitor of IL-18 BP, an antagonist of an activator of IL-18 BP, and/or an agonist of a down-regulator of IL-18 BP. Such a combination therapy may allow for reductions in one or more of the BCG dosage per administration, the number of BCG administrations, the frequency of maintenance BCG therapy, or other opportunities not available for BCG monotherapy, even among subjects responsive to BCG.

If absent or at about the second threshold value or lower, the negatively-correlated biomarker suggests that the level of IL-18 BP may be sufficiently high that the subject would be expected not to respond to BCG monotherapy. Accordingly, to the subject may be administered a therapy selected from (a) (i) BCG and (ii) interleukin 18 (IL-18), an inhibitor of IL-18 BP, an antagonist of an activator of IL-18 BP, and/or an agonist of a down-regulator of IL-18 BP, or (b) a therapy that does not comprise BCG.

If the negatively-correlated biomarker is present, such as at a level above the second threshold value, the biomarker suggests that the level of IL-18 BP may be sufficiently low that the subject would be expected to respond to BCG monotherapy. Accordingly, to the subject may be administered a therapy consisting essentially of BCG. Alternatively, to the subject may be administered a therapy comprising BCG and at least one other therapy. For example, the at least one other therapy may include IL-18, an inhibitor of IL-18 BP, an antagonist of an activator of IL-18 BP, and/or an agonist of a down-regulator of IL-18 BP. Such a combination therapy may allow for reductions in one or more of the BCG dosage per administration, the number of BCG administrations, the frequency of maintenance BCG therapy, or other opportunities not available for BCG monotherapy, even among subjects responsive to BCG.

BCG therapy and therapies that do not comprise BCG will be described in more detail below:

### Bacillus Calmette-Guerin (BCG) Therapy

As stated herein. BCG therapy is the standard of care for non-invasive bladder cancer after initial biopsy/resection of tumor. This may be referred to herein as “primary BCG therapy.” Generally, a liquid formulation containing BCG is administered intravesicularly (e.g., via a transurethral catheter) into the subject's bladder. A typical regimen for BCG administration is once per week for six weeks, followed by a six to twelve week waiting period, after which cystoscopy is performed to evaluate the efficacy of BCG and recurrence of the tumor. Although not to be bound by theory. BCG is generally understood to elicit an immune response against the bacillus, and that immune response is also directed to and can destroy bladder cancer cells.

Also, subjects at high risk of recurrence who are responsive to BCG therapy typically receive maintenance regimens of BCG therapy of about three doses at three, six and then every six months after the primary BCG therapy for up to three years. Such maintenance regimens may be referred to herein as “maintenance BCG therapy.”

Specific aspects of BCG therapy will be known to the person of ordinary skill in the art and need not be described in detail. For example, effective amounts of BCG per dose and per primary or maintenance therapy regimen are known.

### Non-BCG Therapies

The current standard of care for non-invasive bladder cancer involves a number of alternatives to BCG therapy. These alternatives include, but are not necessarily limited to, chemotherapy, radiation therapy, immunotherapy, cystectomy, or combinations thereof. Any of these therapies may be a non-BCG therapy administered to subjects with produced IL-18 BP levels determined to be about a threshold value or higher.

Chemotherapy for non-invasive bladder cancer generally involves at least one dose of a chemotherapeutic administered intravesicularly to the bladder. Commonly-used chemotherapeutic agents include, but are not limited to, mitomycin, gemcitabine, valrubicin, and docetaxel. The chemotherapeutic agent may be heated prior to administration. Alternatively or in addition, an electrical heating apparatus may be inserted intravesicularly to heat the bladder before, during, or after administration of the chemotherapeutic agent.

In other embodiments, systemic chemotherapy may be used. Common chemotherapeutic agents for systemic administration include, but are not limited to, cisplatin, erdafitinib, fluorouracil, mitomycin, gemcitabine, methotrexate, vinblastine, doxorubicin, paclitaxel, and combinations thereof. Particular combinations include, but are not limited to, cisplatin+fluorouracil, fluorouracil+mitomycin, cisplatin+gemcitabine, cisplatin+methotrexate+vinblastine, cisplatin+methotrexate+vinblastine+doxorubicin, and gemcitabine+paclitaxel.

Radiation therapy may involve focusing a beam of radiation on the cancer site. Common radiation therapy regimens include sessions every weekday for 6-12 weeks.

“Immunotherapy” is used herein to refer to the administration of antibodies, antigen-binding fragments, antibody-drug conjugates (ADCs), and the like that bind to one or more proteins in the subject's body, and thereby affect a change in the protein's function that ameliorates the subject's cancer. Examples include, but are not limited to, checkpoint inhibitors, such as atezolizumab, avelumab, nivolumab, and pembrolizumab; ADCs targeted to bladder cancer cells, such as enfortumab vedotin and sacituzumab govitecan.

Cystectomy is the surgical resection of some or all of the patient's bladder.

Any of these therapies or a combination thereof may be chosen by the clinician, based on one or more aspects of the subject's presentation of the cancer, other aspects of the subject's health, one or more demographic factors that may be medically relevant, or one or more genetic markers of the subject, among other parameters.

As should be apparent, additional non-BCG therapies for non-invasive bladder cancer may be developed after this writing. The person of ordinary skill in the art would understand that such therapies could be implemented as a non-BCG therapy in the disclosed method.

Therapy with IL-18, an Inhibitor of IL-18 BP, an Antagonist of an Activator of IL-18 BP, and/or an Agonist of a Down-Regulator of IL-18 BP

The present disclosure also relates to the administration of an effective amount of IL-18, an inhibitor of IL-18 BP, an antagonist of an activator of IL-18 BP, and/or an agonist of a down-regulator of IL-18 BP to the subject. For example, therapy can include BCG and at least one of interleukin 18 (IL-18), an inhibitor of IL-18 BP, an antagonist of an activator of IL-18 BP, and an agonist of a down-regulator of IL-18 BP. In embodiments, administration may be of an effective amount of IL-18 alone: an inhibitor of IL-18 BP alone: an antagonist of an activator of IL-18 BP alone: an agonist of a down-regulator of IL-18 BP alone: IL-18 and an inhibitor of IL-18 BP: IL-18 and an antagonist of an activator of IL-18 BP: IL-18 and an agonist of a down-regulator of IL-18 BP: an inhibitor of IL-18 BP and an antagonist of an activator of IL-18 BP: an inhibitor of IL-18 BP and an agonist of a down-regulator of IL-18 BP: an antagonist of an activator of IL-18 BP and an agonist of a down-regulator of IL-18 BP: IL-18, an inhibitor, and an antagonist: IL-18, an inhibitor, and an agonist: IL-18, an antagonist, and an agonist: an inhibitor, an antagonist, and an agonist; or all of IL-18, an inhibitor, an antagonist, and an agonist.

“IL-18” herein refers to polypeptides comprising the wild type sequences of IL-18 or a cognate molecule of the species of which the subject is a member; polypeptides with one, two, three, four, five, six, seven, eight, nine, ten, or more amino acid insertions, deletions, or substitutions relative to the wild type IL-18 of the species, wherein such polypeptides have from 10% to 1000% of the activity of wild type IL-18; fusion proteins comprising IL-18 covalently bonded, directly or through a linker, to another polypeptide; or other variations in IL-18 that will be known to the person of ordinary skill in the art.

If IL-18 is administered, the administered IL-18 may supplement the subject's autogenous IL-18, thereby changing the balance of IL-18 to IL-18 BP and increasing IFNγ production. By doing so, such as in the subject's bladder by local administration of the IL-18, a therapeutic effect may be observed. For example, the efficacy of BCG therapy may be increased.

The inhibitor of IL-18 BP may inhibit the interaction between IL-18 and IL-18 BP, preferably by binding to IL-18 BP and more preferably by inhibiting the interaction between IL-18 BP and IL-18, e.g., by binding to IL-18 BP and inhibiting the binding of IL-18 BP to IL-18. Examples of such types of inhibitors of IL-18 BP include, but are not limited to, antibodies and antigen-binding fragments that specifically bind IL-18 BP.

The inhibitor of IL-18 BP may reduce IL-18 BP levels, preferably by interfering with transcription or translation of IL-18 BP. Examples of such inhibitors of IL-18 BP include, but are not limited to, antisense oligonucleotides (ASO) and interfering RNAs (RNAis).

The antagonist of an activator of IL-18 BP may disrupt the interaction between the activator and IL-18 BP regulatory elements, if any, preferably by binding to the activator, and more preferably by physically blocking the interaction between the activator and IL-18 BP regulatory elements. If the activator increases the rate of IL-18 BP transcription, the antagonist may disrupt the activator's ability to do so, thereby leading to reduced levels of IL-18 BP. Examples of activators that increase the rate of IL-18 BP transcription include, but are not limited to, transcription factors, nucleosomes, and DNA modifying enzymes. Particular examples include IFN regulatory factor 1 (IRF1) and/or CCAAT/enhancer binding protein β (C/EBPβ).

Conversely, an agonist of a down-regulator of IL-18 BP may bind to the down-regulator and increase its ability to down-regulate IL-18 BP polypeptide activity and/or IL-18 BP transcription, and/or may increase the rate of transcription of the down-regulator.

The reduction in binding of IL-18 BP to IL-18 and/or the reduction in IL-18 BP levels may be systemic or may be local to the subject's bladder. Local administration to the bladder may be performed intravesicularly (e.g., transurethrally). Local administration may be desirable in order to reduce side effects that could arise from an increase in IL-18 activity resulting from systemic IL-18 BP blockade. Such side effects may include, but are not necessarily limited to, hepatitis and arthritis.

An effective amount of IL-18, the inhibitor, the antagonist, or the agonist is an amount that, when administered as part of a therapeutic regimen, is sufficient to mediate a clinically relevant elimination, reduction, amelioration, of the bladder cancer, symptoms thereof, or a relapse thereof under the conditions of administration. An effect is therapeutic if its magnitude is sufficient to impact the health or prognosis of a recipient subject.

The therapeutic effect may occur, or be increased or decreased, in the absence of other treatments or in combination with other treatments. For example, a greater therapeutic effect may occur with administration of an effective amount of IL-18, the inhibitor, the antagonist, and/or the agonist in combination with administration of BCG than may be observed for administering IL-18, the inhibitor, the antagonist, and/or the agonist alone.

An effective amount of IL-18, the inhibitor, the antagonist, and/or the agonist may be generally effective, but may not produce a therapeutic effect in every subject. For example, in scenarios wherein both (a) IL-18, the inhibitor, the antagonist, and/or the agonist and (b) BCG are administered, the rate of response of subjects to BCG may increase from the range of about 50%-70% observed for BCG alone, to a range with a lower bound of about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%. Such a result would indicate both (a) IL-18, the inhibitor, the antagonist, and/or the agonist and (b) BCG are administered in effective amounts, even if the response rate is not 100% across the subject population.

For any particular IL-18, the inhibitor, the antagonist, and/or the agonist, the effective amount may depend on one or more of the mode of action of the molecule, the activity of the molecule, the route of administration, the frequency of administration, the co-administration of other therapies, such as BCG, other parameters of administration (e.g., whether or not the bladder is heated), one or more genetic markers of the subject, one or more demographic markers of the subject, one or more other medical conditions of the subject, and/or subject tolerance for dose-dependent side effects, among others known to and selectable as a routine matter by the person of ordinary skill in the art having the benefit of the present disclosure.

The inhibitor, the antagonist, or the agonist may be selected from numerous molecules and classes of molecules. In embodiments, the inhibitor, the antagonist, or the agonist may be selected from the group consisting of antisense oligonucleotides (ASO) against RNA encoding the IL-18 BP or RNA encoding an activator of IL-18 BP; interfering RNAs (RNAi) therapy against RNA encoding the IL-18 BP or RNA encoding an activator of IL-18 BP; antibody or antigen-binding fragments immunospecifically binding to the IL-18 BP or an activator of IL-18 BP; agonists of a down-regulator of IL-18 BP transcription; polynucleotides for gene therapy to reduce expression of the IL-18 BP by non-cancerous urothelial cells, bladder cancer cells, or both; polynucleotides for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP expression; and combinations thereof. In other words, the inhibitor, antagonist, or agonist may be selected from one, two, three, four, five, six, seven, eight, nine, or all of the inhibitors referred to immediately above. Each of these therapies comprising administration of IL-18, an inhibitor, an antagonist, and/or an agonist will be described in more detail herein.

### Antisense Oligonucleotide (ASO) Therapy

Antisense oligonucleotides (ASOs) are typically 15mer to 50mer DNA polynucleotides that comprising a sequence complementary to at least a target sequence of an RNA of interest, such as an RNA encoding an IL-18 BP, for example, an RNA encoding IL-18 BPa; or an RNA encoding an activator of IL-18 BP. Complementation of the RNA by the ASO may lead to destruction of the RNA, such as by the action of RNase H on the RNA. Destruction of the RNA may lead to reduced rates of translation and/or subsequent reduction in levels of the polypeptide product, in this case, an IL-18 BP. e.g. IL-18 BPa; or an activator of IL-18 BP, which, having its translation rate and/or polypeptide product levels reduced, would lead to reduced activity and/or expression of IL-18 BP.

Alternatively or in addition, complementation of the RNA by the ASO may lead to alternative splicing of the RNA. IL-18 BP has three known isoforms, termed A. B, and C. The A and C isoforms (IL-18 BPa and IL-18 BPc) are believed to reduce IL-18 activity and render BCG therapy less efficacious. The B isoform is not known to impair the efficacy of BCG therapy. Accordingly, alternative splicing of an RNA encoding IL-18 BP may yield an increase in levels of IL-18 BPb, with a corresponding decrease of IL-18 BPa and/or IL-18 BPc levels.

However brought about, reduced levels of IL-18 BP are expected to lead to greater IL-18 activity. In scenarios wherein BCG is also administered as part of the overall therapeutic regimen, this would be expected to lead to improved response to BCG therapy.

The ASO may comprise standard or chemically-modified DNA. Chemical modifications known in the art include, but are not limited to, phosphorothioate internucleoside linkages and modified bases, such as 2′-O)-methoxy-ethyl (2′-MOE) bases and 5-methyl dC in CpG motifs. Such chemical modifications may increase resistance of the ASO to nucleases active against DNA, increase affinity of the ASO for the target sequence, reduce side effects of the ASO therapy, and/or have impart other desirable properties to the ASO as will be known to the person of ordinary skill in the art.

In view of the desirability of local administration of the IL-18, an inhibitor, an antagonist, and/or an agonist over systemic administration, the ASO therapy may comprise intravesicular administration of an ASO complementary to an RNA encoding IL-18 BP (e.g., an RNA encoding IL-18 BPa) or an RNA encoding an activator of IL-18 BP.

### RNA Interference (RNAi) Therapy

RNA interference (RNAi) generally involves the introduction into cells of double stranded RNA (dsRNA) or short hairpin RNA (shRNA). Although the introduced RNAs may be of any length, a range of 300-600 nucleotides (nt) may be effective. Enzymes process these RNAs into short interfering RNA (siRNA) molecules with length about 21 nt, which yield single stranded RNA (ssRNA) which are incorporable into an RNA-induced silencing complex (RISC). The RISC includes endonucleases which may cleave a target mRNA complementary to the ssRNA. Alternatively, or in addition, and especially if the introduced RNA is incompletely complementary to the target mRNA, the target mRNA may form a dsRNA with the ssRNA, wherein the dsRNA cannot be translated into a polypeptide.

Variations of RNAi processes may include introducing siRNAs rather than longer RNAs requiring additional processing, and introducing vectors that express dsRNA, shRNA, or siRNA, among others known to the person of ordinary skill in the art.

Regardless of which mechanism(s) occur for a particular combination of introduced RNA and target mRNA, translation of the target mRNA may be reduced, thereby lowering levels of the polypeptide encoded by the target mRNA. Hence. RNAi therapy against RNA encoding the IL-18 BP may reduce levels of the IL-18 BP (e.g., IL-18 BPa). Similarly. RNAi therapy against an RNA encoding an activator of IL-18 BP may reduce levels of the activator, thereby leading to reduced activity and/or expression of IL-18 BP.

### Antibody or Antigen-Binding Fragment Therapy

Use of an anti-IL-18-BP antibody or antigen-binding fragment, e.g., an anti-IL-18-BPa antibody or antigen-binding fragment, may reduce activity of the IL-18 BP by physically interfering with binding of the IL-18 BP to IL-18 or by reducing IL-18 BP activity, among other avenues that will be known to the person of ordinary skill in the art. It may be the case that an antibody or an antigen-binding fragment against IL-18-BPb or IL-18-BPc (the isoforms of IL-18 BP that that do not bind or bind with less affinity to IL-18, respectively) may be sufficiently cross-reactive with an anti-IL-18-BPa antibody or antigen-binding fragment that it would prove effective in reducing IL-18-BPa activity.

Similarly, use of an antibody or antigen-binding fragment that immunospecifically binds to an activator of IL-18 BP may reduce activity of the activator by physically interfering with binding of the activator to a target molecule, polynucleotide sequence, or polypeptide sequence by which it activates IL-18 BP, or by reducing the activator's activity in other ways, among other avenues that will be known to the person of ordinary skill in the art.

An antibody or antigen-binding fragment that immunospecifically binds to IL-18 at an epitope which leads to physical hindrance of IL-18 BP binding to IL-18 would be expected to reduce IL-18 BP binding to IL-18.

Antibodies and antigen-binding fragments are generally well-known, as are techniques for developing antibodies and antigen-binding fragments against a particular antigen.

However, assuming local, intravesicular administration of an anti-IL-18-BP antibody or antigen-binding fragment, or an antibody or antigen-binding fragment that immunospecifically binds to an activator of IL-18 BP, the antibody or antigen-binding fragment may be cleared more quickly than an ASO or an RNA eliciting an RNAi response.

### IL-18 BP Transcription Down-Regulator Agonist Therapy

Activation of an IL-18 BP transcription down-regulator by an agonist can reduce the levels of IL-18 BP, thereby reducing IL-18 BP's ability to suppress IL-18 activity. Agonists of IL-18 BP transcription down-regulators will depend on the particular IL-18 BP transcription down-regulator being targeted.

### Gene Therapy

Gene therapy is the modification of cells to insert a polynucleotide into cells, e.g., as an episome and/or integrated into the genome, wherein the polynucleotide provides a therapeutic effect, by encoding a polypeptide for which expression is desired, disrupting a genomic sequence encoding a polypeptide which is desired to be silenced, modulating the transcription of an endogenous sequence encoding a polypeptide, or the like. Generally, the modification may be brought about by preparing a vector comprising the polynucleotide and using the vector to insert the polynucleotide into the nuclei of one or more cells.

In embodiments, gene therapy may insert a polynucleotide into non-cancerous urothelial cells, bladder cancer cells, or both, wherein the polynucleotide may lead to reduced expression of an IL-18 BP, e.g. IL-18 BPa. The reduced expression may arise from disrupting the sequence encoding the IL-18 BP, modifying regulatory sequences (e.g., promoters, enhancers, etc.) to reduce the likelihood of transcription of the sequence encoding the IL-18 BP, modifying untranslated regions (UTRs) of an mRNA encoding the IL-18 BP to render the mRNA less likely to be translated, and/or modifying sequences to reduce the proportion of IL-18 BP mRNAs that are spliced to yield IL-18 BPa or IL-18 BPc, among other avenues that will be known to the person of ordinary skill in the art.

Alternatively or in addition, gene therapy may insert a polynucleotide into non-cancerous urothelial cells, bladder cancer cells, or both, wherein the polynucleotide may lead to increased expression of IL-18. The increased expression may arise from increasing the copy number of IL-18 coding sequences in the genome, modifying regulatory sequences to increase the likelihood of transcription of IL-18 coding sequences, modifying UTRs of an IL-18 mRNA to render it more likely to be translated, etc.

As yet another alternative or addition, gene therapy may insert a polynucleotide into non-cancerous urothelial cells, bladder cancer cells, or both, wherein the polynucleotide may increase expression of an inhibitor of IL-18 BP. The increased expression may arise from increasing the copy number of genomic sequences encoding the inhibitor of IL-18 BP, modifying regulatory sequences to increase the likelihood of transcription of coding sequences for the inhibitor of IL-18 BP, modifying UTRs of an mRNA transcript of the inhibitor of IL-18 BP to render it more likely to be translated, etc.

Whether gene therapy increases IL-18 levels, directly or indirectly decreases IL-18 BP levels, or any combination thereof, by increasing the ratio of IL-18 relative to IL-18 BP, a therapeutic effect may be observed. For example, if BCG is also administered as part of the subject's treatment regimen, the efficacy of the BCG therapy may be improved.

Suitable gene therapy vectors are well-known in the art and include, for example AAV vectors, and retroviral vectors (e.g. lentiviral vectors, gamma retroviral vectors).

### Decoy Polynucleotides

A decoy polynucleotide is a polynucleotide which resembles the usual target sequence recognized by a regulatory protein, e.g., a transcription factor. As a result, the decoy polynucleotide can compete with the target sequence, and lower the activity of the regulatory protein with respect to the target sequence. For example, a decoy polynucleotide for a regulatory sequence that promotes or enhances IL-18 BP expression can lead to reduced transcription of the IL-18 BP coding sequence, and hence, lower IL-18 BP levels. For another example, a decoy polynucleotide for a regulatory sequence that lowers expression of an inhibitor of IL-18 BP can lead to increased transcription of the coding sequence of the inhibitor of IL-18 BP, higher levels of the inhibitor of IL-18 BP, and reduced suppression of IL-18 by IL-18 BP. The decoy polynucleotide can be introduced to non-cancerous urothelial cells, bladder cancer cells, or both using vectors and techniques known to the person of ordinary skill in the art.

Aspects Common to Therapies Comprising Administration of IL-18 and/or an Inhibitor of IL-18 BP

Several factors relating to the IL-18 and/or an inhibitor of IL-18 BP are applicable to any of the particular therapies comprising administration of IL-18 and/or an inhibitor of IL-18 BP described herein. For example, any two or more of the therapies comprising administration of IL-18 and/or an inhibitor of IL-18 BP referred to above may be combined. In embodiments, administering may comprise administering IL-18, with or without the co-administration of one, two, three, four, five, six, seven, eight, nine, ten, or more inhibitors of IL-18 BP. In embodiments, administering may comprise administering of one, two, three, four, five, six, seven, eight, nine, ten, or more inhibitors of IL-18 BP, with or without the co-administration of IL-18.

Therapy comprising administration of any IL-18 and/or an inhibitor of IL-18 BP may further comprise heating the bladder. Heating may be performed by use of an electrical heating element introduced via a catheter, by heating a liquid solution comprising a therapeutic agent of the IL-18 and/or an inhibitor of IL-18 BP, or both. Though not to be bound by theory, heating the bladder may enhance uptake of the therapeutic agent into bladder cancer cells, non-cancerous urothelial cells, or both.

If both (a) the IL-18 and/or inhibitor of IL-18 BP and (b) BCG are administered as part of the subject's treatment regimen, one or more doses of the IL-18 and/or inhibitor of IL-18 BP may be administered prior to, essentially simultaneously with, and/or after administering one or more doses of BCG.

For example, prior administration may be helpful to preemptively lower IL-18 BP levels in bladder cells (cancerous cells, non-cancerous urothelial cells, or both), which may permit improved response of the immune system to BCG. Any IL-18 and/or an inhibitor of IL-18 BP may be administered simultaneously with a dose of the BCG therapy.

Alternatively or in addition, essentially simultaneous administration, e.g., placing a transurethral catheter and administering both at least one dose of BCG and at least one dose of the IL-18 and/or inhibitor of IL-18 BP prior to withdrawing the catheter, may be convenient for the clinician, generate higher rates of subject compliance and reduce subject discomfort by combining multiple treatment modalities into a single catheterization, and may reduce IL-18 BP expression induced by IFNγ generated by BCG therapy. Administering both BCG and the IL-18 and/or inhibitor of IL-18 BP may involve sequential administration of BCG followed by IL-18/inhibitor, sequential administration of IL-18/inhibitor followed by BCG, and/or administration of a single formulation containing IL-18/inhibitor and BCG.

If BCG therapy is also provided to the subject, the IL-18 and/or an inhibitor of IL-18 BP need not be administered with every BCG therapy regimen provided to the subject. For example, a subject who was responsive to primary BCG therapy without the performance of a IL-18 and/or an inhibitor of IL-18 BP may, with increasing age, changes in overall health, changes in immune system function, etc. become non-responsive. The IL-18 and/or an inhibitor of IL-18 BP may thus be performed with maintenance BCG therapy.

As should be apparent, if BCG therapy is performed as part of the subject's treatment regimen, the IL-18 and/or an inhibitor of IL-18 BP may be administered in conjunction with primary BCG therapy, both primary and maintenance BCG therapy, and/or maintenance BCG therapy.

Any IL-18 and/or an inhibitor of IL-18 BP may be administered between doses of a BCG therapy and/or after completion of a BCG therapy regimen, if such is performed.

The IL-18 and/or an inhibitor of IL-18 BP may involve one or multiple doses. The dose(s) of the IL-18 and/or an inhibitor of IL-18 BP may be administered at any desired time(s) during the subject's treatment regimen.

Any of the therapies comprising administration of IL-18 and/or an inhibitor of IL-18 BP may involve the formulation and administration of a pharmaceutical composition comprising a therapeutically-effective amount of an active ingredient (i.e., IL-18 and/or an inhibitor of IL-18 BP) and one or more pharmaceutically-acceptable excipients. Particular excipients for particular active ingredients, e.g. for IL-18 and/or any of the various classes of inhibitors of IL-18 BP that may be used, can be selected by the person of ordinary skill in the art having the benefit of the present disclosure as a routine matter.

The pharmaceutical composition may, but need not, further comprise one or more active ingredients other than IL-18 and/or an inhibitor of IL-18 BP. For example, the pharmaceutical composition may further comprise BCG.

### Interleukin 15 (IL-15) Agonist Therapy

Interleukin 15 (IL-15) is a cytokine that recruits natural killer cells. Activation of IL-15 by an agonist may thus minimize the detrimental impact of IL-18-BP in bladder cancer therapy. e.g. BCG therapy, by routing around it to a different signaling pathway leading to recruitment of components of the immune system to attack bladder cancer cells. Such attack may be concomitant with an attack on BCG, if BCG therapy is also performed as part of the subject's treatment regimen. Use of IL-15 agonists may thus supplement therapy with IL-18, an inhibitor, an antagonist, and/or an agonist. In general, agonists of IL-15 will be known to the person of ordinary skill in the art and need not be described in detail.

### Interferon Therapy

We have observed that IFNγ induces IL-18 BP expression in both bladder cancer cells and non-cancerous urothelial cells in a quasi-dose-dependent manner, e.g., exposing cells to increasing concentrations of IFNγ generally leads to higher expression of IL-18 BP. However, administration of one or more other IFNs may activate one or more signaling pathways leading to recruitment of components of the immune system to attack bladder cancer cells. Such attack may be concomitant with an attack on BCG, if BCG therapy is also performed as part of the subject's treatment regimen. Any IFN (other than IFNγ) or a combination thereof may be used. For example, for human subjects, the IL-18 and/or an inhibitor of IL-18 BP may comprise administration of IFNα, IFNβ, IFNε, IFNκ, IFNω, IFNλ, or two or more thereof. Use of interferons other IFNγ may thus supplement therapy with IL-18 and/or inhibitors of IL-18 BP.

### First and Second Medical Uses

IL-18 therapy, ASO therapy. RNAi therapy, antibody therapy, antigen-binding fragment therapy, gene therapy, and or decoy polynucleotide therapy involve one or more compounds. The disclosure relates to use of any compound described herein in therapy, such as therapy for bladder cancer, including bladder cancer not otherwise responsive to BCG treatment. The disclosure also relates to any use of any compound described herein for the manufacture of a medicament.

### Identification of BCG Responders and Non-Responders

The compositions comprising IL-18, an inhibitor, an antagonist, and/or an agonist may be used, optionally with BCG, without regard to whether the subject is or is expected to be responsive or non-responsive to BCG monotherapy (meaning a therapy consisting essentially of BCG and lacking other therapeutic modalities, including IL-18 or inhibitors of IL-18 BP). However, it may be desirable to identify the subject as being non-responsive to a monotherapy consisting of BCG, prior to administering IL-18, an inhibitor, an antagonist, and/or an agonist; administering BCG, or both.

### Third Therapies

Even after administering IL-18, an inhibitor, an antagonist, and/or an agonist, with or without administering BCG, a subject's bladder cancer may not be fully eliminated. The current standard of care for non-invasive bladder cancer involves a number of alternatives or follow-ons to BCG therapy. These same alternatives or follow-ons may be performed after administering BCG therapy and the IL-18, an inhibitor, an antagonist, and/or an agonist according to the present disclosure. These alternatives include, but are not necessarily limited to, chemotherapy, radiation therapy, non-BCG immunotherapy, cystectomy, or combinations thereof.

Chemotherapy for non-invasive bladder cancer generally involves at least one dose of a chemotherapeutic administered intravesicularly to the bladder. Commonly-used chemotherapeutic agents include, but are not limited to, mitomycin, gemcitabine, valrubicin, and docetaxel. The chemotherapeutic agent may be heated prior to administration. Alternatively or in addition, an electrical heating apparatus may be inserted intravesicularly to heat the bladder before, during, or after administration of the chemotherapeutic agent.

In other embodiments, systemic chemotherapy may be used. Common chemotherapeutic agents for systemic administration include, but are not limited to, cisplatin, erdafitinib, fluorouracil, mitomycin, gemcitabine, methotrexate, vinblastine, doxorubicin, paclitaxel, and combinations thereof. Particular combinations include, but are not limited to, cisplatin+fluorouracil, fluorouracil+mitomycin, cisplatin+gemcitabine, cisplatin+methotrexate+vinblastine, cisplatin+methotrexate+vinblastine+doxorubicin, and gemcitabine+paclitaxel.

Radiation therapy may involve focusing a beam of radiation on the cancer site. Common radiation therapy regimens include sessions every weekday for 6-12 weeks.

“Immunotherapy” is used herein to refer to the administration of antibodies, antigen-binding fragments, antibody-drug conjugates (ADCs), and the like that bind to one or more proteins in the subject's body, and thereby affect a change in the protein's function that ameliorates the subject's cancer. Examples include, but are not limited to, checkpoint inhibitors, such as atezolizumab, avelumab, nivolumab, and pembrolizumab; and ADCs targeted to bladder cancer cells, such as enfortumab vedotin and sacituzumab govitecan.

Cystectomy is the surgical resection of some or all of the patient's bladder.

Any of these therapies or a combination thereof may be chosen by the clinician, based on one or more aspects of the subject's presentation of the cancer, other aspects of the subject's health, one or more demographic factors that may be medically relevant (e.g., the subject's age, race, or sex), or one or more genetic markers of the subject, among other parameters.

As should be apparent, additional non-BCG therapies for non-invasive bladder cancer may be developed after this writing. The person of ordinary skill in the art would understand that such therapies could be implemented as a non-BCG therapy in the disclosed method.

### Kits

Instructions for performing any method disclosed herein may be packaged with one or more of the reagents or components used in that method. Such a packaging of the instructions and the reagents or components may be termed a “kit.”

The instructions may be packaged in the kit in the form of printed instructions, a printed document providing a uniform resource locator (URL) from which detailed instructions may be accessed upon a user's entry of the URL into the address bar of a web browser, a printed document providing a Quick Response (QR) code which can be scanned to direct a smartphone or tablet computer's browser to a URL, or the like.

Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as Styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

One kit of the present disclosure may comprise instructions to quantify the expression of an IL-18 BP (e.g., IL-18 BPa) by performing RT-qPCR on RNA encoding IL-18 BP, and RT-qPCR primers for the amplification of RNA encoding IL-18 BP (e.g., RNA encoding IL-18 BPa).

Another kit of the present disclosure may comprise instructions to quantify the expression of IL-18 BP by performing an enzyme-linked immunosorbent assay (ELISA) on IL-18 BP, and an ELISA testing unit comprising an antibody against IL-18 BP (e.g., against IL-18 BPa) of the species of which the subject is a member.

One kit of the present disclosure may comprise instructions to administer to a subject in need thereof an effective amount of interleukin 18 (IL-18), an inhibitor of IL-18 BP, an antagonist of an activator of IL-18 BP, and/or an agonist of a down-regulator of IL-18 BP. The instructions may further comprise instructions to administer the IL-18, the inhibitor, the antagonist, and/or the agonist by intravesical administration to the bladder. The instructions may additionally include instructions to heat the bladder. The instructions may additionally comprise instructions to administer bacillus Calmette-Guérin (BCG) to the subject in need thereof; or wherein the subject in need thereof has received or will receive BCG. The instructions may call for the BCG therapy as a primary BCG therapy or a maintenance BCG therapy. Instructions to administer BCG may comprise instructions to administer BCG by intravesical administration to the bladder. The instructions may comprise instructions to administer the IL-18, inhibitor, antagonist, and/or agonist essentially simultaneously with the BCG. The instructions may comprise, prior to the administering, identifying the subject as being non-responsive to BCG monotherapy. The instructions may additionally include instructions to administer a third therapy other than IL-18, the inhibitor, the antagonist, and/or the agonist (and BCG, if BCG is administered), such as after administering IL-18, the inhibitor, the antagonist, and/or the agonist (and BCG, if BCG is administered). The instructions may call for the third therapy to be chemotherapy, radiation therapy, non-BCG immunotherapy, cystectomy, or combinations thereof.

This kit may include a composition, comprising an effective amount of interleukin 18 (IL-18), an inhibitor of IL-18 BP, an antagonist of an activator of IL-18 BP, and/or an agonist of a down-regulator of IL-18 BP, and a pharmaceutically-acceptable carrier. In the composition, the inhibitor, the antagonist, and/or the agonist may be selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP expression; agonists of a down-regulator of IL-18 BP transcription; a polynucleotide for gene therapy to reduce expression of IL-18 BP by non-cancerous urothelial cells, bladder cancer cells, or both: a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both: a decoy polynucleotide for a regulatory sequence that promotes or enhances IL-18 BP; and combinations thereof. For example, the inhibitor may be an ASO complementary to an RNA encoding IL-18 BP isoform A (IL-18 BPa). In a particular example, the ASO may comprise a sequence such that complementation of the RNA by the ASO reduces translation of the RNA, reduces splicing of the RNA to yield IL-18 BPa or IL-18 BPc, or both. The composition may further comprise an effective amount of bacillus Calmette-Guérin (BCG). The composition may be formulated for intravesical administration to the bladder.

In an embodiment, IFNγ induces IL-18BP expression. In an embodiment, TNFα can potentiate IFNγ induction of IL-18BP.

In an embodiment, IFNγ can decrease uroplakin 1b up to 2 fold. In an embodiment, TNFα can decrease uroplakin 1b up to 2 fold. In an embodiment, IFNγ+TNFα can decrease uroplakin 1b up to 5 fold.

In an embodiment, IL-18BP/uroplakin 1b is higher on exposure to IFNγ in higher risk patients. In an embodiment, IL-18BP/uroplakin 1b shows no change on exposure to TNFα in higher risk patients. In an embodiment, IL-18BP/uroplakin 1b is lower on exposure to IFNγ+TNFα in higher risk patients. In an embodiment, the ratio of IFNγ to IFNγ+TNFα is a predictor of recurrence and/or progression. In an embodiment, uroplakin 1b is likely not decreasing upon exposure to IFNγ+TNFα.

### ILLUSTRATIVE EMBODIMENTS

### Group 1

1-1. A method for treating bladder cancer, comprising administering to a subject in need thereof an effective amount of interleukin 18 (IL-18), an inhibitor of IL-18 binding protein (IL-18 BP), an antagonist of an activator of IL-18 BP, and/or an agonist of a down-regulator of IL-18 BP.

1-2. The method of embodiment 1-1, wherein the inhibitor, antagonist, and/or agonist reduces binding of IL-18 BP to IL-18 in the bladder or reduces IL-18 BP levels in the bladder.

1-3. The method of any one of the preceding embodiments, wherein the IL-18, the inhibitor, the antagonist, and/or the agonist is/are administered by intravesical administration to the bladder.

1-4. The method of any one of the preceding embodiments, wherein the inhibitor, the antagonist, or the agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP expression; agonists of a down-regulator of IL-18 BP transcription: a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

1-5. The method of any one of the preceding embodiments, wherein the administering comprises intravesicular administration of an ASO complementary to an RNA encoding IL-18 BP isoform A (IL-18 BPa), to an RNA encoding IL-18 BP isoform C (IL-18 BPc), or to an RNA encoding the activator of IL-18 BP.

1-6. The method of any one of embodiments 1-4 and 1-5, wherein the ASO comprises a sequence that, upon complementation of the RNA encoding IL-18 BP or the activator thereof by the ASO, reduces translation of the RNA and/or alters splicing of the RNA encoding IL-18 BP, thereby reducing the yield of IL-18 BPa and/or IL-18 BPc.

1-7. The method of any one of the preceding embodiments, further comprising heating the bladder.

1-8. The method of any one of the preceding embodiments, further comprising administering bacillus Calmette-Guerin (BCG) to the subject in need thereof; or wherein the subject in need thereof has received or will receive BCG.

1-9. The method of embodiment 1-8, wherein the BCG is administered by intravesical administration to the bladder.

1-10. The method of any one of embodiments 1-8 and 1-9, wherein the IL-18, the inhibitor, the antagonist, and/or the agonist is/are administered essentially simultaneously with the BCG.

1-11. The method of any one of embodiments 1-8-1-10, wherein the BCG therapy is a primary BCG therapy or a maintenance BCG therapy.

1-12. The method of any one of the preceding embodiments, further comprising identifying the subject as being non-responsive to BCG monotherapy prior to administering the IL-18, the inhibitor, the antagonist, and/or the agonist.

1-13. The method of any one of the preceding embodiments, wherein the inhibitor of IL-18 BP reduces the binding of IL-18 BP isoform A (IL-18 BPa) and/or IL-18 BP isoform C (IL-18 BPc) to IL-18 and/or reduces levels of IL-18 BPa and/or IL-18 BPc.

1-14. The method of any one of the preceding embodiments, further comprising, after administering IL-18, the inhibitor, the antagonist, and/or the agonist (and BCG, if BCG is administered), administering a third therapy other than IL-18, the inhibitor, the antagonist, and/or the agonist (and BCG, if BCG is administered).

1-15. The method of embodiment 1-14, wherein the third therapy is selected from the group consisting of chemotherapy, radiation therapy, non-BCG immunotherapy, cystectomy, and combinations thereof.

1-16. The method of any one of embodiments 1-14 and 1-15, wherein the third therapy is chemotherapy.

1-17. A composition, comprising: an effective amount of interleukin 18 (IL-18), an inhibitor of IL-18 binding protein (IL-18 BP), an antagonist of an activator of IL-18 BP, and/or an agonist of a down-regulator of IL-18 BP, and a pharmaceutically-acceptable excipient.

1-18. The composition of embodiment 1-17, wherein the composition is formulated for intravesical administration to the bladder.

1-19. The composition of any one of the embodiments 1-17 and 1-18, wherein the inhibitor, antagonist, or agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both: a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

1-20. The composition of any one of embodiments 1-17-1-19, wherein the inhibitor is an ASO complementary to an RNA encoding IL-18 BP isoform A (IL-18 BPa) and/or IL-18 BP isoform C (IL-18 BPc) or to an RNA encoding the activator of IL-18 BP.

1-21. The composition of any one of embodiments 1-19-1-20, wherein the ASO comprises a sequence such that complementation of the RNA encoding IL-18 BP or the activator thereof by the ASO reduces translation of the RNA and/or alters splicing of the RNA encoding IL-18 BP, thereby reducing the yield of IL-18 BPa and/or IL-18 BPc.

1-22. The composition of any one of embodiments 1-17-1-21, further comprising an effective amount of bacillus Calmette-Guérin (BCG).

1-23. A kit, comprising: instructions for performing the method of any one of embodiments 1-1-1-16, and a composition comprising an effective amount of interleukin 18 (IL-18), an inhibitor of IL-18 binding protein (IL-18 BP), an antagonist of an activator of IL-18 BP, and/or an agonist of a down-regulator of IL-18 BP, and a pharmaceutically-acceptable carrier.

1-24. The kit of embodiment 1-23, wherein the inhibitor reduces binding of IL-18 BP to IL-18 in the bladder, reduces IL-18 BP levels in the bladder, and/or activates an immune pathway not mediated by IL-18 in the bladder.

1-25. The kit of embodiment 1-23, wherein the composition is formulated for intravesical administration to the bladder.

1-26. The kit of any one of the embodiments 1-23-1-25, wherein the inhibitor of IL-18 BP is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP expression; and combinations thereof.

1-27. The kit of any one of embodiments 1-23-1-26, wherein the inhibitor is an ASO complementary to an RNA encoding IL-18 BP isoform A (IL-18 BPa) and/or IL-18 BP isoform C (IL-18 BPc) or to an RNA encoding the activator of IL-18 BP expression.

1-28. The kit of any one of embodiments 1-26-1-27, wherein the ASO comprises a sequence such that complementation of the RNA encoding IL-18 BP or the activator thereof by the ASO reduces translation of the RNA and/or alters splicing of the RNA encoding IL-18 BP, thereby reducing the yield of IL-18 BPa and/or IL-18 BPc.

1-29. The kit of any one of embodiments 1-23-1-28, wherein the composition further comprises an effective amount of bacillus Calmette-Guérin (BCG).

### Group 2

2-1. A method for selecting a subject for bladder cancer therapy, comprising:

providing a sample from a subject with bladder cancer; determining from the sample the presence, absence, or level of a biomarker that correlates with interleukin 18 binding protein (IL-18 BP) expression by bladder cells from the subject; and when the biomarker positively correlates with IL-18 BP expression and the biomarker is present or has a level at or above a first predetermined threshold, or when the biomarker negatively correlates with IL-18 BP expression and the biomarker is absent or has a level at or below a second predetermined threshold, the subject is identified as a candidate for a therapy selected from (a) (i) bacillus Calmette-Guérin (BCG) and (ii) interleukin 18 (IL-18), an inhibitor of IL-18 BP, an antagonist of an activator of IL-18 BP, and/or an agonist of a down-regulator of IL-18 BP or (b) a therapy that does not comprise BCG.

2-2. The method of embodiment 2-1, wherein the biomarker is IFN regulatory factor 1 (IRF1) and/or CCAAT/enhancer binding protein β (C/EBPβ).

2-3. The method of any one of the preceding embodiments, wherein the biomarker is a single nucleotide polymorphism (SNP), such as an SNP within or near the IL-18 BP transcriptional promoter.

2-4. The method of any one of the preceding embodiments, wherein when the biomarker positively correlates with IL-18 BP expression and the biomarker is absent or has a level at or below the first predetermined threshold, or when the biomarker negatively correlates with IL-18 BP expression and the biomarker is present or has a level at or above the second predetermined threshold, the subject is identified as a candidate for therapy consisting essentially of BCG.

2-5. The method of any one of the preceding embodiments, wherein determining the presence, absence, or level of the biomarker comprises: isolating urothelial cells from the sample, wherein the sample is a bladder biopsy; and determining the protein level and/or the RNA level of interleukin 18 binding protein (IL-18 BP) or a polypeptide biomarker, the IL-18 BP or the polypeptide biomarker produced by the isolated cells.

2-6. The method of embodiment 2-5, further comprising: normalizing the protein level and/or the RNA level of IL-18 BP or the polypeptide biomarker.

2-7. The method of embodiment 2-6, wherein normalizing is to the protein level and/or RNA level of at least one gene (i) selective for urothelium and not bladder muscle and (ii) not regulated by IFNγ, for example, a uroplakin.

2-8. The method of any one of the preceding embodiments, further comprising: exposing urothelial cells of the sample to IFNγ prior to determining the presence, absence, or level of the biomarker.

2-9. The method of embodiment 2-8, wherein exposing the urothelial cells to IFNγ comprises maintaining the urothelial cells in a culture medium comprising from 0.1 ng/mL IFNγ to 500 ng/ml IFNγ.

2-10. The method of any one of embodiments 2-8-2-9, wherein the first threshold value is from 5 μg/mL IL-18 BP secreted in up to 2 mL of culture medium per 100,000 cells after 48 hours of exposure to 1 ng/ml or more of IFNγ to 1000 μg/mL secreted in up to 2 mL of culture medium IL-18 BP per 100,000 cells after 48 hours of exposure to 1 ng/ml or more of IFNγ.

2-11. The method of embodiment 2-10, wherein the first threshold value is 20 μg/mL IL-18 BP secreted in up to 2 mL of culture medium per 100,000 cells after 48 hours of exposure to 1 ng/ml or more of IFNγ.

2-12. The method of any one of embodiments 2-8-2-11, wherein the cells are maintained in the culture media for about 48 hours.

2-13. The method of any one of embodiments 2-5-2-12, wherein determining the protein level and/or the RNA level of IL-18 BP comprises determining the protein level and/or RNA level of one or more IL-18 BP isoforms.

2-14. The method of any one of embodiments 2-5-2-13, wherein determining the RNA level of the IL-18 BP comprises performing reverse-transcriptase quantitative PCR (RT-qPCR) on RNA encoding IL-18 BP.

2-15. The method of any one of embodiments 2-5-2-13, wherein determining the protein level of the IL-18 BP comprises performing an enzyme-linked immunosorbent assay (ELISA) on IL-18 BP.

2-16. The method of any one of the preceding embodiments, wherein the therapy that does not comprise BCG is selected from the group consisting of chemotherapy, radiation therapy, immunotherapy, cystectomy, and combinations thereof.

2-17. The method of any one of the preceding embodiments, wherein the therapy that does not comprise BCG is chemotherapy.

2-18. The method of any one of the preceding embodiments, further comprising: administering to the subject the therapy selected from (a) (i) bacillus Calmette-Guérin (BCG) and (ii) interleukin 18 (IL-18), an inhibitor of IL-18 BP, an antagonist of an activator of IL-18 BP, and/or an agonist of a down-regulator of IL-18 BP or (b) a therapy that does not comprise BCG.

2-19. A kit, comprising: instructions for performing the method of any one of embodiments 2-1-2-14 and 2-16-2-18, and RT-qPCR primers for the amplification of RNA encoding IL-18 BP.

2-20. A kit, comprising: instructions for performing the method of any one of embodiments 2-1-2-13 and 2-15-2-18, and an ELISA testing unit comprising an antibody against IL-18 BP of the species of which the subject is a member.

2-21. The kit of embodiment 2-20, wherein the antibody against IL-18 BP is specific for IL-18 BPa.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

The following are examples of methods and compositions of the invention. It is understood that various other embodiments may be practiced, given the general description provided herein.

### A. Example 1. Bladder Cancer Cell Lines Produce the Th1-Inhibitory Protein IL-18 BPa but not the Th1-Inhibitory Protein IL-12p40 on Exposure to IFNγ

This example describes the production of IL-18 BPa by bladder cancer cells.

### 1.1. Methods

Six cell lines were harvested, counted, and seeded in 12 well plates in medium containing 10% FCS. The cell lines were: T24 high grade bladder cancer. TCCsup high grade bladder cancer. HT-1197 high grade bladder cancer. RT4 low grade bladder cancer. 5637 low grade bladder cancer, and SW780 low grade bladder cancer.

LNCaP prostate cancer cells were used as a control.

After 24 hrs. the medium was replaced, and the cells were exposed to four different concentrations of IFNγ: 0, 1, 10 and 50 ng/ml.

Conditioned medium and cells were harvested after 48 hrs and IL-12p70. IL-12p40. IL-18, and IL-18 BPa were measured using commercially available ELISAs. IL-18BPa mRNA was measured using two step real time PCR with normalization to GPDH mRNA levels. Experiments were carried out three times in duplicate.

RNA was extracted from the cancer cell lines using the RNeasy Mini Kit (Qiagen. Valencia. CA) following the manufacturer's instructions. RNA was eluted in nuclease-free water and stored at −20° C. RNA concentration and quality was assessed using a NanoDrop™ 2000 spectrophotometer (Thermo Fisher Scientific. Waltham. MA). RNA was converted to cDNA using the High-Capacity cDNA Reverse Transcription Kit (Applied Biosystems. Foster City. CA) following the manufacturer's guidelines. cDNA concentration and quality were also verified through spectrophotometry. PCR amplification was performed using the TaqMan™ Fast Advanced Master Mix (Applied Biosystems. Foster City. CA) and TaqMan assays for IL-18BP (Hs04981047_s1: GGCAGCAGCTGCTTCGGATCCACAC) (SEQ ID NO:6) and GAPDH (Hs02786624_g1: CGCTGCCAAGGCTGTGGGCAAGGTC) (SEQ ID NO:7) as a housekeeping gene according to the manufacturer's recommendations. The _s1 denotes an assay with primers and probes designed within a single exon. The _g1 indicates an assay that may detect genomic DNA, with primers and probes that may be within a single exon. The PCR program was as follows: enzyme activation for 2 min at 95° C., and 40 cycles of denaturation for 20 s at 95° C., and annealing/extension for 30 s at 60° C. Delta delta Ct (ΔΔCt) values were calculated and normalized.

Over 75% confluent 100 mm plates were treated with recombinant IFNγ, R&D Systems, Minneapolis, MN, at a final concentration of 50 ng/ml and incubated for 48 hours. Conditioned media was collected filtered using 0.45 μm filters and concentrated to 1 ml/plate using 10 kDa molecular weight cut-off (MWCO) filters.

SDS-PAGE for concentrated cell conditioned media corresponding to the cancer cell lines was run in a Novex™ WedgeWell™ 4-20% Tris-Glycine gel from Invitrogen (Thermo Fisher Scientific, Waltham, MA) using a Xcell SureLock™ from Invitrogen (Thermo Fisher Scientific, Waltham, MA) and % X Tris Glycine Running Buffer. Gel was washed and soaked in ethanol for 5 min. After soaking, the gel was transferred using iBlot 2R and iBlot 2R PVDF Mini Stacks from Invitrogen (Thermo Fisher Scientific, Waltham, MA) for 7 min (1 min at 20V, 4 min at 23V and the remaining time at 25V). Immunodetection was performed employing 1:250 mouse anti-IL18BPa antibody (MAB1191, R&D Systems, Minneapolis, MN) as the primary antibody and 1:5000 goat anti-mouse IgG/IgM-HRP (Sigma-Aldrich, St. Louis, MO) as the coupled secondary antibody. Briefly, the membrane was blocked under shaking in 5% non-fat dry milk in phosphate buffered saline (PBS 1×) for 30 min followed by 30 min blocking in 5% filtered bovine serum albumin (BSA) fraction V from Roche (Sigma-Aldrich, St. Louis, MO) in PBS 1×. After completing the blocking steps, the membrane was incubated under shaking for 1 hr at room temperature with the primary antibody, washed with 0.1% Tween 20 PBS 1× and finally incubated under shaking for 1 hr at room temperature with the secondary antibody. The membrane was then washed with 0.1% Tween 20 PBS 1× and the immunocomplexes were detected colorimetrically after 3,3′,5,5′-Tetramethylbenzidine (TMB) substrate addition.

### 1.2. Results

All six human bladder cancer cell lines expressed IL-18 BPa at >20 pg/ml over 48 hrs per 100,000 cells of serum-containing medium in vitro when exposed to IFNγ.

In all lines, we observed a trend towards increasing expression of IL-18 BPa protein and mRNA with exposure to greater concentrations of IFNγ.

No IL-12p40 monomer, the other Th1-inhibitory cytokine, could be detected in vitro with or without IFNγ in any of the bladder cancer cell lines.

LNCaP prostate cancer cells were used as a control. LNCaP showed minimal expression of each cytokine in media with serum, and consistent expression of only IL-12p40 in serum free media regardless of IFNγ concentration from 0-50 ng/ml.

### 1.3. Conclusions

Preliminary experiments with six human bladder cancer cell lines suggest that IL-18 BPa and not IL-12p40, the other Th-1 inhibitory cytokine, may be produced by bladder cancer upon exposure to IFNγ in vivo.

As expected for IFNγ-inducing cytokines, only minimal amounts of IL-18 and no IL-12p70 were detected in vitro with or without IFNγ (almost 100×less than IL-18 BPa at 50 ng/ml IFNγ) in all bladder cancer cell lines.

IL-18 BPa expression by bladder cancer may result in immune modulation that favors cancer cell survival by decreasing BCG-induced IFNγ.

### B. Example 2. Non-Cancerous Urothelial Cells Produce IL-18 BPa but not IL-12p40 on Exposure to IFNγ

This example describes the production of IL-18 BPa by non-cancerous urothelial cells.

A Th1-type immune response to BCG characterized by elevated production of IFNγ has shown to be effective against non-muscle invasive bladder cancer (NMIBC). Macrophage and other antigen presenting cell-produced IL-12 and IL-18 mediate Th1 polarization. IL-18 was originally termed IFNγ inducing factor. IL-18 cannot induce Th 1 cell development as can IL12, but has the capacity to activate established Th1 cells to produce IFNγ in the presence of IL-12. IL-18 Binding Protein, specifically isoforms a and c (hIL-18BPa and hIL-18BPc) in humans, and isoforms c and d (mIL-18BPc and mIL-18BPd) in mice, inhibit the activity of IL-18 by binding to IL-18 and blocking its interaction with the IL-18 receptor. The balance between Th1 and Th2 polarizing cytokines, is thought to explain the varied response to BCG in bladder cancer. Changes in IL-18BPa levels in urine, as a response to BCG treatment, have been shown to predict disease recurrence and/or progression. In an effort to understand how normal urothelial cells respond to IFNγ, we measured IL-18, IL-12p70, IL-18BPa and IL-12p40 levels in the culture medium of normal human urothelial cells after exposure to IFNγ. With the goal of evaluating the suitability of murine models for IL-18BP modulation, we tested the response of cultured normal mouse urothelial cells to IFNγ, TNFα, and IL-6, alone or in combination.

### 2.1. Methods

Non-cancerous urothelial cells were harvested, counted, and seeded in 12 well plates in serum free, growth factor supplemented medium.

Normal primary human urothelial cells from the American Type Culture Collection (ATCC) were harvested, counted, and seeded in 12 well plates in serum free medium (Bladder Epithelial Cell Basal Medium, catalog #PCS-420-032, ATCC, Manassas, VA) containing: 5 μg/ml insulin, 5 ng/ml EGF, 1 μM epinephrine, 5 μg/ml Apo-transferrin, 0.5 ng/ml TGFα, 0.4% bovine pituitary extract (Extract P™, Lifeline Cell Technology, Frederick, MD), 100 ng/ml hydrocortisone, and 5 ng/ml KGF.

After 24 hrs, the medium was replaced, and the cells are exposed to 4 different concentrations of IFNγ: 0, 1, 10 and 50 ng/ml.

Conditioned medium was harvested after 48 hrs and IL-12p70, IL-12p40, IL-18, and IL-18 BPa are measured using commercially available ELISAs. Experiments were carried out twice, in duplicate.

Normal mouse urothelial cells from C57BL/6, Balb/c mice and the mouse bladder cancer cell line MBT-2 were harvested, counted, and seeded in 12 well plates in serum containing medium. After 24 hrs, the medium was replaced, and the cells were exposed to IFNγ, TNFα, and IL-6, alone or in combination.

Normal bladder biopsies were obtained from patients (n=3) with bladder cancer under an IRB approved protocol. Biopsies were split into roughly equal sections and transferred one per well in a 24 well plate containing 1 ml of serum-free DMEM unless otherwise noted. One well was supplemented with 10 ng/ml IFNγ. After 24 hrs, the tissue was transferred to −80° C. for subsequent mRNA extraction and RT-qPCR for uroplakin and IL-18BPa.

RT-qPCR followed the protocol of Example 1, with the addition of a TaqMan assay for Hs01041715_m1 (uroplakin 1b (urothelium specific). IL18BPa ΔΔCt values were calculated and normalized relative to GAPDH adjusted for uroplakin 1b.

### 2.2. Results

Non-cancerous urothelial cells expressed IL-18 BPa at >200 pg/ml over 48 hrs per 100,000 cells in serum-containing medium in vitro when exposed to IFNγ at 10 ng/ml of greater.

hIL-18BPa expression at >20 μg/ml was observed over 48 hrs per 400,000 cells in 2 ml of serum free medium in vitro when exposed to IFNγ.

A trend towards increasing expression of IL-18 BPa with exposure to greater concentrations of IFNγ was observed.

No IL-12p40 monomer, the other Th1-inhibitory cytokine, was detected in vitro with or without IFNγ in non-cancerous urothelial cells.

The results are summarized in Table 1:

TABLE 1

| | IFNγ | | | |
|---|---|---|---|---|
| ATCC normal cells | 0 ng/ml | 1 ng/ml | 10 ng/ml | 50 ng/ml |
| IL-18BPa (pg/ml) | 0* | 435* | 773* | 472* |
| IL-12p40 (pg/ml) | 0 | 0 | 0 | 0 |
| IL-12p70 (pg/ml) | 0 | 0 | 0 | 0 |
| IL-18 (pg/ml) | <10 pg/ml | <10 pg/ml | <10 pg/ml | <10 pg/ml |

*Average of 2 experiments done in duplicate

Neither mIL-18BPc nor mIL-18BPd could be detected in the medium of normal mouse urothelial cells from C57BL/6 or Balb/c mice or the mouse bladder cancer cell line MBT-2 on exposure to IFNγ, TNFα, and IL-6, alone or in combination.

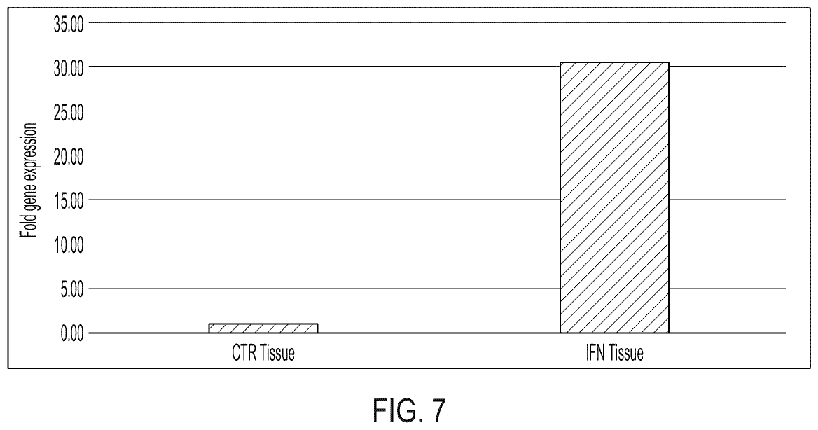

Induction of hIL-18BPa was noted on exposure of normal appearing mucosa of bladder cancer patients in vitro to IFNγ at 10 ng/ml for 24 and 48 hrs. FIG. 7 shows that IL-18 BPa mRNA was induced by IFNγ in fresh normal-appearing bladder biopsy from a patient with bladder cancer. The tissue was treated with 10 ng/ml IFNγ for 48 hrs at 37° C. in Bladder Epithelial Cell Basal Medium under ambient containing CO. IFNγ induced a roughly 30-fold increase in IL-18BPa mRNA.

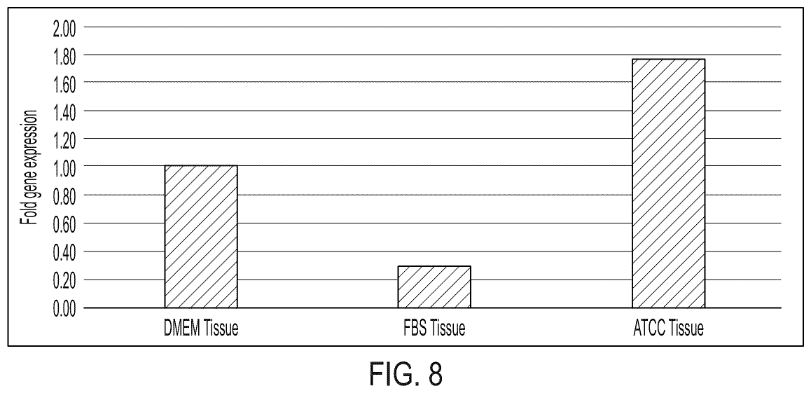

Other media, specifically, serum-free DMEM and DMEM with 10% fetal calf serum (a.k.a. fetal bovine serum, FBS), were tested, with results shown in FIG. 8. In fresh normal-appearing bladder biopsy from a patient with bladder cancer, IL-18 BPa mRNA induction by 10 ng/ml IFNγ for 6 hrs, 37° C., under ambient containing CO, was most pronounced for Bladder Epithelial Cell Basal Medium (ATCC), intermediate for serum-free DMEM, and lowest for DMEM supplemented with FBS.

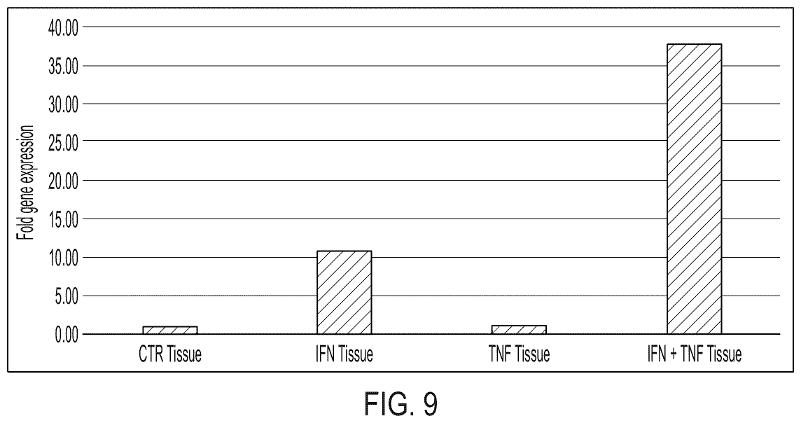

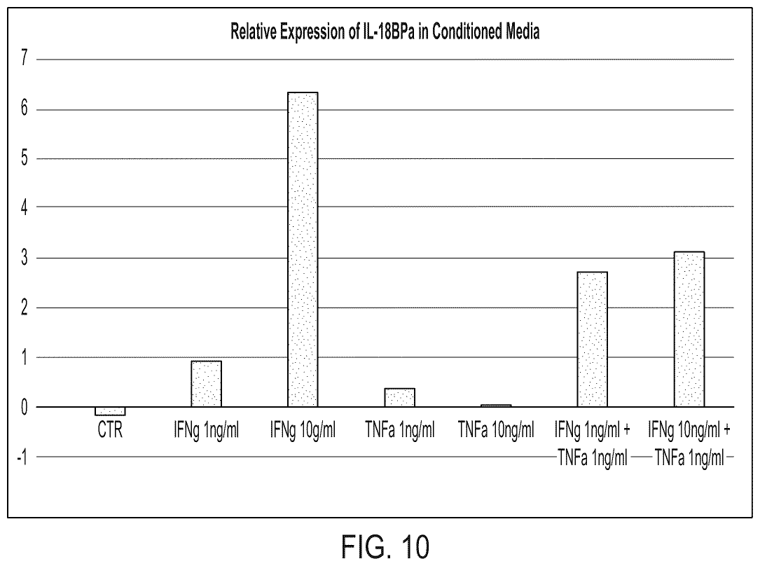

FIG. 9 shows that IFNγ and TNF may have a synergistic effect on IL-18 BPa mRNA induction in fresh normal-appearing bladder biopsy from a patient with bladder cancer. 10 ng/ml IFNγ alone induced about a 10-fold increase in IL-18 BPa mRNA. 10 ng/ml TNF alone had an effect comparable to baseline. A combination of 10 ng/ml IFNγ and 10 ng/ml TNF induced over a 35-fold increase in IL-18 BPa mRNA. All exposure to IFNγ and/or TNF was for 24 hrs in serum-free DMEM (37° C., under ambient containing CO).

### 2.3. Conclusions

Non-cancerous urothelial cells produce IL-18 BPa and not IL-12p40, the other Th-1 inhibitory cytokine, upon exposure to IFNγ in vivo.

Minimal amounts of IL-18 and IL-12p70 were detected in vitro with or without IFNγ.

Preliminary experiments with normal appearing bladder biopsies from patients with bladder cancer indicate that hIL-18BPa is induced upon exposure to IFNγ.

IL-18 BPa expression by non-cancerous urothelial cells may result in immune modulation that favors cancer cell survival by decreasing BCG-induced IFNγ.

### C. Example 3. The IL-18 Neutralizing Activity of Conditioned Medium from IFNγ Exposed Bladder Cancer Cells Lines can be Blocked with an Anti-IL-18BPa Antibody

This example shows that conditioned medium from IFNγ exposed bladder cancer cell lines can neutralize the bioactivity of recombinant IL-18 and that the neutralizing activity is at least in part mediated by IL-18BPa.

### 3.1. Methods

Near confluent 100 mm plates of six bladder cancer cell lines (T24, TCCsup, HT-1197, RT4, 5637, and SW780) were exposed to IFNγ at 100 ng/ml for 48 hrs and the conditioned medium was harvested.

The medium was filtered through a 0.45 μm filter and concentrated 10:1 with a 10K MWCO centrifugal filter.

50,000 HEK-Blue IL-18 cells (Invivogen) expressing the IL-18 receptor and SEAP under the control of the NF-κB and AP-1 pathways were plated per well in 96 well plates supplemented with either: 1) conditioned medium from one of the bladder cancer cell lines exposed to IFNγ, 2) recombinant human IL-18, 3) conditioned medium from one of the bladder cancer cell lines exposed to IFNγ+recombinant human IL-18, or 4) conditioned medium from one of the bladder cancer cell lines exposed to IFNγ+recombinant human IL-18+anti-IL-18BPa antibody.

After 24 hrs the HEK-Blue IL-18 cell conditioned medium was harvested and SEAP activity was assayed.

### 3.2. Results

Conditioned medium from IFNγ exposed bladder cancer cell lines neutralized the bioactivity of recombinant IL-18 in the HEK-Blue IL-18 cell assay.

The IL-18 neutralizing activity of the conditioned medium from IFNγ exposed bladder cancer cell lines was partially blocked by an anti-IL-18BPa antibody.

### 3.3. Conclusions

A significant fraction, if not all of the IL-18 neutralizing activity of the conditioned medium from all six bladder cancer cell lines exposed to IFNγ is mediated by IL-18BPa expressed by the bladder cancer cell lines.

### D. Example 4. Improved Response to BCG Therapy by Administering ASO Therapy in an In Vivo Mouse IL-18BPa Transgenic Mouse Model

This example describes an in vivo assessment of the impact of ASO therapy on BCG response in a mouse model.

### 4.1. Methods

A mouse model commonly used for bladder cancer studies is acquired, maintained, and handled according to the established American Association for Accreditation of Laboratory Animal Care (AAALAC) protocols. A bladder cancer cell line is established in each mouse following published protocols. Administration of BCG therapy to the mice follows published protocols. The mouse model has a first rate of response to BCG monotherapy, i.e., BCG therapy in the absence of any therapy with an IL-18 BP inhibitor.

A control group of mice receives BCG monotherapy and an experimental group receives BCG therapy and ASO therapy targeting IL-18 BP. The two groups are substantially matched in age, sex, diet, maintenance, and handling.

After the completion of the control or the experimental therapy regimen, the mice are humanely euthanized, the bladders dissected, and tumor size and/or weight measured for each animal.

### 4.2. Results

The control group has an average control tumor size and an average control tumor weight. The experimental group has an average experimental tumor size and an average experimental tumor weight.

The average tumor size and weight are both less in the experimental group than the control group.

A number of animals experience a reduction of the tumor to an undetectable extent in the experimental group. This number is greater than the number of animals in the control group who experience such a reduction of the tumor.

### 4.3. Conclusions

In the mouse model, BCG therapy in conjunction with ASO therapy targeting IL-18 BP provides an improved responsiveness rate of mice with bladder cancer to BCG therapy.

This result suggests BCG therapy in conjunction with ASO therapy targeting IL-18 BP would provide an improved responsiveness rate of humans with bladder cancer to BCG therapy.

### E. Example 5. In Vivo Assessment of the Impact of siRNA, ASO and Anti-hIL-18BPa Antibody Therapy on BCG Response in a IL-18BPa Transgenic Mouse Model

This example describes an in vivo assessment of the impact of siRNA, ASO and anti-hIL-18BPa antibody therapy on BCG response in a IL-18BPa transgenic mouse model.

### 5.1. Methods

A transgenic C3H/He mouse for human IL-18BPa is generated by injecting the human IL-18BPa DNA fragment into oocytes to create transgenic embryos that are transplanted into pseudo-pregnant FVB/N mothers. Mice positive by Southern blot analysis are back-crossed into the C3H/He background for at least six generations and handled according to the established American Association for Accreditation of Laboratory Animal Care (AAALAC) protocols. MBT-2 bladder cancer cells are implanted orthotopically following published protocols. Administration of BCG therapy to the mice follows published protocols. The mouse model has a first rate of response to BCG monotherapy, i.e., BCG therapy in the absence of any therapy with an IL-18 BP inhibitor.

A control group of mice receives BCG monotherapy and experimental groups receive BCG therapy and ASO therapy targeting IL-18 BP or siRNA therapy targeting IL-18 BP or anti-hIL-18BPa therapy targeting IL-18 BP. The groups are substantially matched in age, sex, diet, maintenance, and handling.

After the completion of the control or the experimental therapy regimen, the mice are humanely euthanized, the bladders dissected, and tumor size and/or weight measured for each animal as well as any metastases.

### 5.2. Results

The control group has an average control tumor size and an average control tumor weight. The experimental group has an average experimental tumor size and an average experimental tumor weight.

The average tumor size and weight are both less in the experimental group than the control group.

A number of animals experience a reduction of the tumor to an undetectable extent in the experimental group. This number is greater than the number of animals in the control group who experience such a reduction of the tumor.

### 5.3. Conclusions

In the mouse model, BCG therapy in conjunction with either of the three therapies targeting IL-18 BP provides an improved responsiveness rate of mice with bladder cancer to BCG therapy.

This result suggests BCG therapy in conjunction with therapy targeting IL-18 BP would provide an improved responsiveness rate of humans with bladder cancer to BCG therapy.

### F. Example 6. Effect of IFNγ and TNFα, Alone or in Combination, on the Expression of Uroplakin 1B and IL-18BPa by Normal Urothelial Cells in Culture

### 6.1 Methods

2×10\hat{ }5 normal urothelial cells (LifeLine Celltech, Inc.) were plated in each of 7 wells in a 12 well culture treated plate in serum free complete LifeLine Celltech growth medium. After 24 hrs, the medium was replaced with 2 ml of new serum free complete LifeLine Celltech growth medium and one well each was treated with, 1) no cytokine. 2) 1 ng/ml IFNγ. 3) 10 ng/ml IFNγ, 4) 1 ng/ml IFNγ. 5) 10 ng/ml IFNγ, 6) 1 ng/ml TNFα+10 ng/ml IFNγ. After 48 hrs, the medium and cells were harvested. The medium was clarified by centrifugation and tested for expression of IL-12p40, IL-18, IL-18BPa, and IL-12p70 by ELISAs (DY1240, DY318-05, DY119, and DY1270, R & D Systems, Inc). RNA was extracted from the cells using silica columns (RNeasy, Qiagen, Inc.). RNA samples were quantified in a Nanodrop 2000 (Thermo Scientific, Inc.). mRNAs were converted to cDNA using High Capacity cDNA Reverse Transcription Kit (Applied Biosystems, Inc.) Using the 7500 Fast Real Time PCR System (Applied Biosystems, Inc.), real time PCR was carried out with the following protocol: Enzyme Activation at 95° C., for 2 min once followed by 40 cycles of: denaturation at 95° C., for 20 sec and annealing and extension at 60° C., for 30 sec. The following Taqman Gene Expression assays were used: Hs01041715_m1 for uroplakin 1b-FAM, Hs04981047_s1 for IL18BP-FAM, Hs02786624_g1 for GAPDH-FAM and ROX as a passive reference. The ΔΔCt method was used to calculate cDNA levels.

### 6.2 Results

IL-12p40, IL-18, and IL-12p70 as measured by ELISA in conditioned medium were below the level of detection in all samples. IL-18BPa concentration in the medium increased with exposure to IFNγ and, less markedly with TNFα. Exposure to the combination of 1 ng/ml of IFNγ with 1 ng/ml of TNFα increased IL-18BPa concentration in the medium more than exposure to either alone.

As shown in Table 2, total mRNA harvested from each well was reduced only in the wells containing cells treated with the combination of IFNγ and TNFα, reflecting a decreased cell number. As expected, the threshold cycle for the housekeeping gene GAPDH was higher only in the wells containing cells treated with the combination of IFNγ and TNFα. IL-18BPa mRNA increased with exposure to IFNγ and, less markedly with TNFα relative to the housekeeping gene GAPDH. Exposure to the combination of 1 ng/ml of IFNγ with 1 ng/ml of TNFα increased IL-18BPa mRNA more than exposure to either alone. Uroplakin 1B mRNA decreased with exposure to IFNγ and TNFα relative to the housekeeping gene GAPDH. Exposure to the combination of IFNγ and TNFα decreased Uroplakin 1B mRNA more than exposure to either alone.

TABLE 2

| | | | IL-18BPa | Uroplakin |
|---|---|---|---|---|
| | | | mRNA | 1B mRNA |
| | | | level | level |
| | RNA | GAPDH | relative to | relative to |
| | Concentration | Cycle | GAPDH | GAPDH |
| Sample ID | (ng/ul) | Threshold | (2\hat{ }-(ΔΔCt)) | (2\hat{ }-(ΔΔCt)) |
| Blank | | | | |
| CTR | 65.2 | 17.14 | 1.00 | 1.00 |
| IFNg 1 ng/ml | 67.1 | 17.04 | 1.29 | 0.75 |
| IFNγ 10 ng/ml | 61.9 | 17.29 | 16.58 | 0.48 |
| TNFα 1 ng/ml | 57.0 | 17.29 | 1.18 | 0.63 |
| TNFα 10 ng/ml | 62.7 | 17.44 | 1.24 | 0.72 |
| IFNγ 1 ng/ml + | 48.0 | 17.73 | 3.44 | 0.19 |
| TNFα 1 ng/ml | | | | |
| IFNγ 10 ng/ml + | 34.9 | 18.42 | 6.19 | 0.09 |
| TNFα 1 ng/ml | | | | |

### G. Example 7. Correlation Between AUA Risk Group (https://www.auanet.org/guidelines-and-quality/guidelines/bladder-cancer-non-muscle-invasive-guideline) or EORTC recurrence risk (https://www.omnicalculator.com/health/eortc-bladder-cancer) and IL-18BPa mRNA Change Relative to Uroplakin 1B on Exposure of Normal Urothelium to Either IFNγ, TNFα or the Combination

### 7.1 Methods

Normal appearing urothelial tissue was obtained at the time of tumor resection using a biopsy forceps and placed in a sterile container on ice and transferred to the lab. The tissue was split into 4 fragments and each fragment cultured in separate wells of a 12 well plate. Tissues were treated with either nothing (CTR), IFNγ 10 ng/ml or TNFα 10 ng/ml or IFNγ 10 ng/ml+TNFα 10 ng/ml×24 hrs at 37 C in 5% CO in serum free DMEM medium. After 24 hrs, RNA was extracted from the tissues using silica columns (RNeasy, Qiagen, Inc.). RNA samples were quantified in a Nanodrop 2000 (Thermo Scientific, Inc.). mRNAs were converted to cDNA using High Capacity cDNA Reverse Transcription Kit (Applied Biosystems, Inc.) Using the 7500 Fast Real Time PCR System (Applied Biosystems, Inc.), real time PCR was carried out with the following protocol: Enzyme Activation at 95 C for 2 min once followed by 40 cycles of: denaturation at 95° C., for 20 sec and annealing and extension at 60 C for 30 sec. The following Taqman Gene Expression assays were used: Hs01041715_m1 for uroplakin 1b-FAM, Hs04981047_s1 for IL18BP-FAM and ROX as a passive reference. The ΔΔCt method was used to calculate cDNA levels relative to Uroplakin 1B correcting for the CTR sample mRNA levels. Table 3.

TABLE 3

| | | | IL-18BPa/Uroplakin 1B (2\hat{ } ΔΔCt) | | | |
|---|---|---|---|---|---|---|
| | EAU | EUA | | | | IFNγ/ |
| AUA | progression | recurrence | | | IFNγ + | (IFNγ + |
| risk | risk | risk | IFNγ | TNFα | TNFα | TNFα) |
| 1 | 2 | 2 | 5.67 | 1.26 | 55.22 | 0.100 |
| 1 | 2 | 2 | 6.04 | 0.97 | 35.93 | 0.168 |
| 2 | 2 | 3 | 10.79 | 1.18 | 37.66 | 0.286 |
| 3 | 14 | 10 | 13 | 1.33 | 14.93 | 0.871 |
| 3 | 13 | 5 | 15 | 1.51 | 10.66 | 1.407 |
| 3 | 10 | 9 | 30.92 | 1.59 | 24.94 | 1.240 |

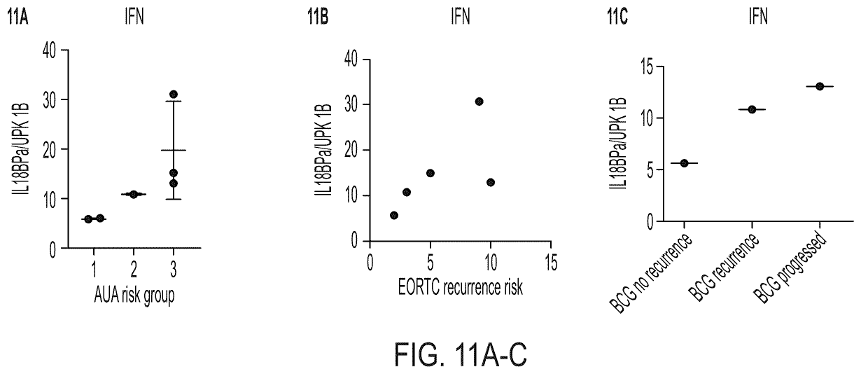

FIG. 11A-11C shows IL-18BPa mRNA change relative to Uroplakin 1B on exposure of normal urothelium to IFNγ.

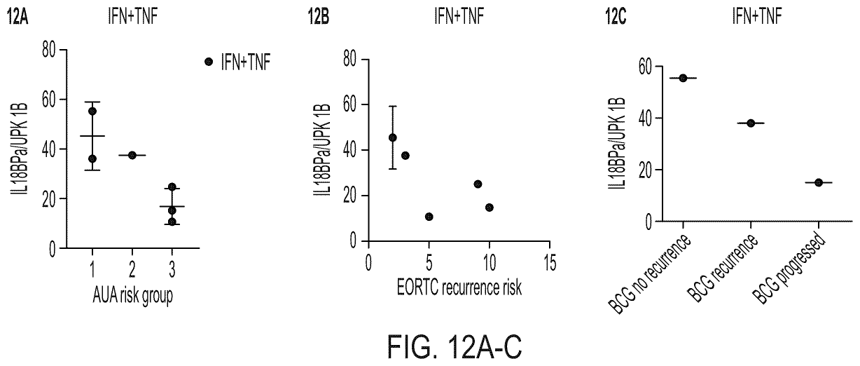

FIG. 12A-12C shows IL-18BPa mRNA change relative to Uroplakin 1B on exposure of normal urothelium to the combination of IFNγ and TNFα.

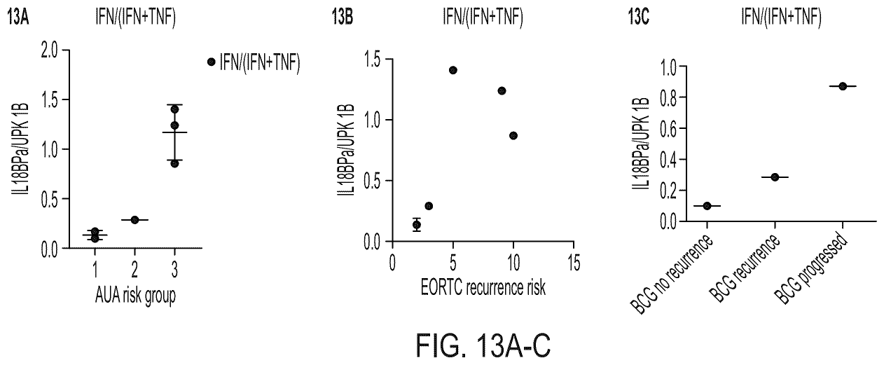

FIG. 13A-13C shows IL-18BPa mRNA change relative to Uroplakin 1B on exposure of normal urothelium to the ratio of IFNγ/IFNγ+TNFα.

### H. Example 8. siRNA Against IL-18BPa and Double Stranded DNA Transcription Factor Decoy Against IRF-1, but not Against STAT1 Mediated Knockdown of IL-18BPa in T24 Bladder Cancer Cells in Culture

### 8.1 Methods

siRNA assays SASI_Hs01_00161669 and SASI_Hs01_00161670 were purchased from Sigma. STAT1 decoy (sequence=[+C] [+A]CTTACAGGAATATGCATAACA [+T][+G] where [ ] are locked nucleic acid modified bases) (SEQ ID NO:8) and IRF-1 decoy (sequence=[+G][+G]AAGCGAAAATGAAATTGA [+C][+T] where [ ] are locked nucleic acid modified bases) (SEQ ID NO:9) were purchased from Sigma. 1×10\hat{ }6 bladder cancer cells were plated in each of 6 wells in a 12 well culture treated plate in 10% Fetal Serum DMEM medium. After 24 hrs, the medium was replaced and one well each was treated with, 1) no treatment, 2) Lipofectamine 3000, 3) IL-18BPa siRNA (SASI_Hs01_00161669)+Lipofectamine 3000, 4) IL-18BPa siRNA (SASI_Hs01_00161670)+Lipofectamine 3000, 5) IRF1-decoy+Lipofectamine 3000, 6) STAT1-decoy+Lipofectamine 3000. After 24 hrs, the medium was replaced and IFNγ at 10 ng/ml final concentration was added to all of the wells except 1. After 48 hrs, the medium was harvested. The medium was clarified by centrifugation and tested for expression of IL-18BPa using a commercially available ELISA assay (DY119, R & D Systems, Inc).

### 8.2 Results

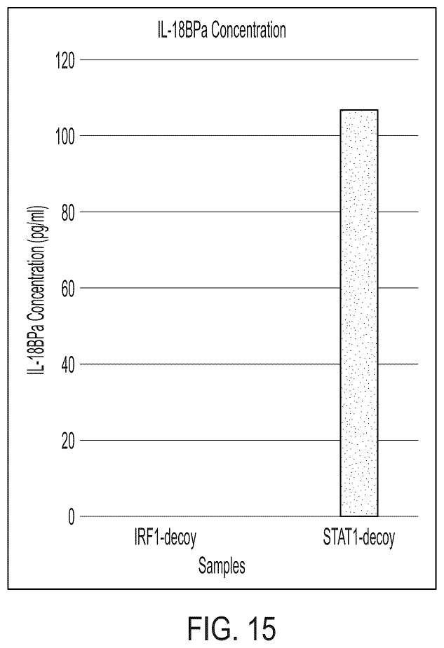

IL-18BPa concentration in the medium increased with exposure to IFNγ (compare wells 1 and 2). Transfection with either SASI_Hs01_00161669 or SASI_Hs01_00161669 siRNAs directed against IL-18BPa decreased expression of IL-18BPa by greater than 4 fold. Exposure to either the IRF-1 or the STAT1 transcription factor decoys was noted to result a decrease in the number of harvested cells by 50%. As shown in FIG. 15, the IRF-1 directed decoy resulted in compete elimination of IL-18BPa expression while the STAT1 decoy did not.

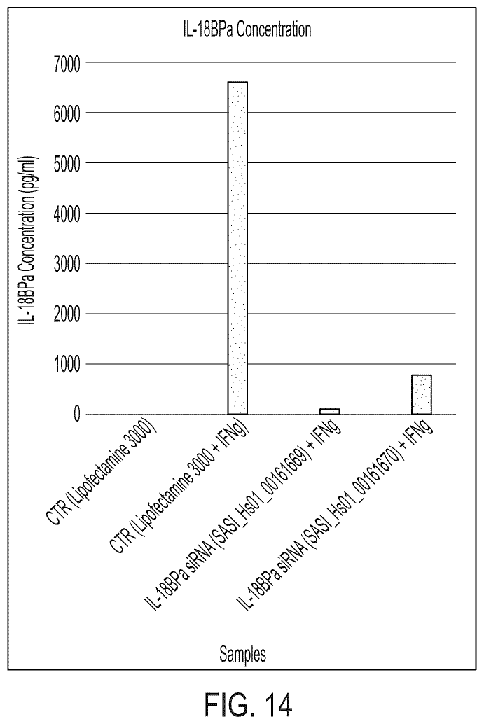

FIG. 14 shows IL-18BPa concentration in the medium upon exposure IFNγ or siRNAs directed against IL-18BPa.

FIG. 15 shows IL-18BPa concentration in the medium upon exposure to IRF-1 or STAT1 transcription factor decoys.

### 7. REFERENCES

1. Takeda K, Tsutsui H, Yoshimoto T, Adachi O, Yoshida N, Kishimoto K, et al. Defective NK cell activity and Th1 response in IL-18-deficient mice. Immunity 1998; 8:383-90.
2. Dinarello C A, Targeting interleukin-18 with interleukin-18 binding protein. Annals of the Rheumatic Diseases 2000:59:117-120
3. Herr H W, Morales A. History of bacillus Calmette-Guerin and bladder cancer: an immunotherapy success story. J Urol. (2008) 179:53-6. doi: 10.1016/j.juro.2007.08.122
4. Yi Luo, Eric J. Askeland, Mark R. Newton, Jonathan R. Henning and Michael A. O'Donnell (May 9, 2013). Immunotherapy of Urinary Bladder Carcinoma: BCG and Beyond, Cancer Treatment-Conventional and Innovative Approaches, Letícia Rangel, IntechOpen, DOI: 10.5772/55283. Available from: https://www.intechopen.com/chapters/44365.
5. Amirali Salmasi, David A Elashoff, Rong Guo, Alexander Upfill-Brown, Charles J Rosser, Jason M Rose, Louise C Giffin, Louis E Gonzalez, Karim Chamie. Cancer Epidemiol Biomarkers Prev. 2019 June: 28 (6): 1036-1044. doi: 10.1158/1055-9965.EPI-18-0893. Available from: https://pubmed.ncbi.nlm.nih.gov/30593457/.
6. G N Thalmann, A Sermier, C Rentsch, K Mohrle, M G Cecchini, U E Studer. J Urol. 2000 December: 164 (6): 2129-33. Available from: https://pubmed.ncbi.nlm.nih.gov/11061941/.
7. Y-J Kim, Y-S Ha, S-K Kim, H. Y. Yoon, M. S. Lym, M-J Kim, S-K Moon, Y. H. Choi, and W-J Kim, Clin. Cancer Res. 2010 16 (7) 2131-37.

### 8. SEQUENCES

| Seq. | | |
|---|---|---|
| No. | Description | Sequence |
| 1 | Human IL-18 | MAAEPVEDNCINFVAMKFID |
| | | NTLYFIAEDDENLESDYFGK |
| | | LESKLSVIRNLNDQVLFIDQ |
| | | GNRPLFEDMTDSDCRDNAPR |
| | | TIFIISMYKDSQPRGMAVTI |
| | | SVKCEKISTLSCENKIISFK |
| | | EMNPPDNIKDTKSDIIFFQR |
| | | SVPGHDNKMQFESSSYEGYF |
| | | LACEKERDLFKLILKKEDEL |
| | | GDRSIMFTVQNED |
| | | (SEQ ID NO: 1) |
| 2 | Human IL-18BPa | MTMRHNWTPDLSPLWVLLLC |
| | | AHVVTLLVRATPVSQTTTAA |
| | | TASVRSTKDPCPSQPPVFPA |
| | | AKQCPALEVTWPEVEVPLNG |
| | | TLSLSCVACSRFPNFSILYW |
| | | LGNGSFIEHLPGRLWEGSTS |
| | | RERGSTGTQLCKALVLEQLT |
| | | PALHSTNFSCVLVDPEQVVQ |
| | | RHVVLAQLWAGLRATLPPTQ |
| | | EALPSSHSSPQQQG |
| | | (SEQ ID NO: 2) |
| 3 | Human Uroplakin | MAKDNSTVRCFQGLLIFGNV |
| | | IIGCCGIALTAECIFFVSDQ |
| | | H1BSLYPLLEATDNDDIYGA |
| | | AWIGIFVGICLFCLSVLGIV |
| | | GIMKSSRKILLAYFILMFIV |
| | | YAFEVASCITAATQQDFFTP |
| | | NLFLKQMLERYQNNSPPNND |
| | | DQWKNNGVTKTWDRLMLQDN |
| | | CCGVNGPSDWQKYTSAFRTE |
| | | NNDADYPWPRQCCVMNNLKE |
| | | PLNLEACKLGVPGFYHNQGC |
| | | YELISGPMNRHAWGVAWFGF |
| | | AILCWTFWVLLGTMFYWSRI |
| | | EY |
| | | (SEQ ID NO: 3) |
| 4 | Human IFNgamma | MKYTSYILAFQLCIVLGSLG |
| | | CYCQDPYVKEAENLKKYFNA |
| | | GHSDVADNGTLFLGILKNWK |
| | | EESDRKIMQSQIVSFYFKLF |
| | | KNFKDDQSIQKSVETIKEDM |
| | | NVKFFNSNKKKRDDFEKLTN |
| | | YSVTDLNVQRKAIHELIQVM |
| | | AELSPAAKTGKRKRSQMLFR |
| | | GRRASQ |
| | | (SEQ ID NO: 4) |
| 5 | Human TNFalpha | MSTESMIRDVELAEEALPKK |
| | | TGGPQGSRRCLFLSLFSFLI |
| | | VAGATTLFCLLHFGVIGPQR |
| | | EEFPRDLSLISPLAQAVRSS |
| | | SRTPSDKPVAHVVANPQAEG |
| | | QLQWLNRRANALLANGVELR |
| | | DNQLVVPSEGLYLIYSQVLF |
| | | KGQGCPSTHVLLTHTISRIA |
| | | VSYQTKVNLLSAIKSPCQRE |
| | | TPEGAEAKPWYEPIYLGGVF |
| | | QLEKGDRLSAEINRPDYLDF |
| | | AESGQVYFGIIAL |
| | | (SEQ ID NO: 5) |
| 6 | IL-18BP | GGCAGCAGCTGCTTCGGATC |
| | (Hs04981047_s1) | CACAC |
| | | (SEQ ID NO: 6) |
| 7 | GAPDH | CGCTGCCAAGGCTGTGGGC |
| | (Hs02786624_g1) | AAGGTC |
| | | (SEQ ID NO: 7) |
| 8 | STAT1 decoy | [+C][+A]CTTACAGGAATA |
| | | TGCATAACA[+T][+G] |
| | | (SEQ ID NO: 8) |
| 9 | IRF-1 decoy | [+G][+G]AAGCGAAAATG |
| | | AAATTGA[+C][+T] |
| | | (SEQ ID NO: 9) |

1. A method for determining treatment for bladder cancer, comprising
a) providing bladder cells from a subject with bladder cancer;
b) culturing at least a portion of the cells with IFNγ and/or culturing at least a portion of the cells with IFNγ and TNFα, each culture maintained for a period of time sufficient to induce expression of interleukin 18 binding protein (IL-18 BP) by the cells;
c) determining expression level of IL-18 BP and uroplakin 1B by the cells cultured in b); and
determining the ratio of expression level of IL-18 BP to uroplakin 1b for cells cultured with IFNγ (IL-18BP:uroplakin 1b IFNγ) and/or for cells cultured with IFNγ and TNFα (IL-18BP:uroplakin 1b IFNγ+TNFα); wherein a IL-18BP:uroplakin 1b IFNγ of about 6.0 or greater and/or a IL-18BP:uroplakin 1b IFNγ+TNFα of about 40 or lower indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy.

2. (canceled)

3. The method of claim 1, wherein determining the expression level in c) comprises determining at least one of a protein level and a mRNA level.

4. (canceled)

5. The method of claim 1, wherein IL-18BP:uroplakin 1b IFNγ and IL-18BP:uroplakin 1b IFNγ+TNFα is determined.

6. (canceled)

7. The method of claim 1, wherein the inhibitor, the antagonist, or the agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP expression; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

8. The method of claim 1, wherein the inhibitor of IL-18BP is an anti-IL18BP antibody.

9. The method of claim 1, further comprising administering the therapy to the subject with bladder cancer.

10. The method of claim 1, wherein the therapy is administered by intravesical administration to the bladder.

11. The method of claim 1, further comprising heating the bladder.

12. The method of claim 1, wherein at least one of the inhibitor, antagonist, and agonist reduces binding of IL-18 BP to IL-18 in the bladder or reduces IL-18 BP levels in the bladder.

13. The method of claim 9, wherein the administering comprises intravesicular administration of an ASO complementary to an RNA encoding IL-18 BP isoform A (IL-18 BPa), to an RNA encoding IL-18 BP isoform C (IL-18 BPc), or to an RNA encoding the activator of IL-18 BP.

14. The method of claim 13, wherein the ASO comprises a sequence that, upon complementation of the RNA encoding IL-18 BP or the activator thereof by the ASO, causes at least one of reduces translation of the RNA and alters splicing of the RNA encoding IL-18 BP, thereby reducing the yield of IL-18 BPa and/or IL-18 BPc.

15. The method of claim 1, further comprising administering bacillus Calmette-Guérin (BCG) to the subject in need thereof; or wherein the subject in need thereof has received or will receive BCG.

16. (canceled)

17. (canceled)

18. (canceled)

19. The method of claim 1, wherein the inhibitor of IL-18 BP reduces the binding of IL-18 BP isoform A (IL-18 BPa) and/or IL-18 BP isoform C (IL-18 BPc) to IL-18 and/or reduces levels of IL-18 BPa and/or IL-18 BPc.

20. The method of claim 1, wherein the non-BCG therapy is selected from the group consisting of chemotherapy, radiation therapy, immunotherapy, cystectomy, and combinations thereof.

21. A composition, comprising: at least one of an effective amount of interleukin 18 (IL-18), an inhibitor of IL-18 binding protein (IL-18 BP), an antagonist of an activator of IL-18 BP, and an agonist of a down-regulator of IL-18 BP, and a pharmaceutically-acceptable excipient.

22. The composition of claim 21, wherein the composition is formulated for intravesical administration to the bladder.

23. The composition of claim 21, wherein the inhibitor, antagonist, or agonist is selected from the group consisting of antisense oligonucleotides (ASO) against RNA encoding IL-18 BP or RNA encoding an activator of IL-18 BP; interfering RNAs (RNAi) against RNA encoding IL-18 BP or RNA encoding an activator of IL-18 BP; antibodies or antigen-binding fragments thereof immunospecifically binding to IL-18 BP or an activator of IL-18 BP; agonists of a down-regulator of IL-18 BP transcription; a polynucleotide for gene therapy to reduce expression of IL-18 BP by non-cancerous urothelial cells, bladder cancer cells, or both; a polynucleotide for gene therapy to increase expression of IL-18 by non-cancerous urothelial cells, bladder cancer cells, or both; a polynucleotide for gene therapy to increase expression of an inhibitor of IL-18 BP by non-cancerous urothelial cells, bladder cancer cells, or both; a decoy polynucleotide for a regulatory sequence that promotes or enhances IL-18 BP; and combinations thereof.

24. The composition of claim 21, further comprising an effective amount of bacillus Calmette-Guérin (BCG).

26. (canceled)

27. (canceled)

28. (canceled)

29. A method for selecting a subject for bladder cancer therapy, comprising:
a) providing a sample from a subject with bladder cancer;
b) determining from the sample the presence, absence, or level of a biomarker that correlates with interleukin 18 binding protein (IL-18 BP) expression by bladder cells from the subject; and wherein when the biomarker positively correlates with IL-18 BP expression and the biomarker is present or has a level at or above a first predetermined threshold, or when the biomarker negatively correlates with IL-18 BP expression and the biomarker is absent or has a level at or below a second predetermined threshold, the subject is identified as a candidate for a therapy selected from (a) (i) bacillus Calmette-Guérin (BCG) and (ii) at least one of interleukin 18 (IL-18), an inhibitor of IL-18 BP, an antagonist of an activator of IL-18 BP, and an agonist of a down-regulator of IL-18 BP or (b) a therapy that does not comprise BCG.

30. The method of claim 29, wherein the biomarker is at least one of IFN regulatory factor 1 (IRF1) and CCAAT/enhancer binding protein β (C/EBPβ).

31. A method for determining treatment for bladder cancer, comprising
a) providing bladder cells from a subject with bladder cancer;
b) culturing a portion of the cells with IFNγ and separately culturing a portion of the cells with IFNγ and TNFα, each culture maintained for a period of time sufficient to induce expression of interleukin 18 binding protein (IL-18 BP) and/or IL-18 by the cells;
c) determining expression level of IL-18 BP and uroplakin 1B by the cells cultured in b);
d) determining the ratio of expression level of IL-18 BP to uroplakin 1b for cells cultured with IFNγ (IL-18BP:uroplakin 1b IFNγ) and for cells cultured with IFNγ and TNFα (IL-18BP:uroplakin 1b IFNγ+TNFα); and
e) determining the (IL-18BP:uroplakin 1b IFNγ):(IL-18BP:uroplakin 1b IFNγ+TNFα) ratio, wherein a (IL-18BP:uroplakin 1b IFNγ):(IL-18BP:uroplakin 1b IFNγ+TNFα) ratio of about 0.2 or greater indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy.

32. (canceled)

33. The method of claim 31, wherein determining the expression level in c) comprises determining a protein level and/or a mRNA level.

34. (canceled)

35. (canceled)

36. (canceled)

37. The method of claim 31, further comprising administering the therapy to the subject with bladder cancer.

38. (canceled)

39. (canceled)

40. The method of claim 31, wherein the at least one of the inhibitor, antagonist, and agonist reduces binding of IL-18 BP to IL-18 in the bladder or reduces IL-18 BP levels in the bladder.

41. The method of claim 37, wherein the administering comprises intravesicular administration of an ASO complementary to an RNA encoding IL-18 BP isoform A (IL-18 BPa), to an RNA encoding IL-18 BP isoform C (IL-18 BPc), or to an RNA encoding the activator of IL-18 BP.

42. The method of claim 41, wherein the ASO comprises a sequence that, upon complementation of the RNA encoding IL-18 BP or the activator thereof by the ASO, causes at least one of reduces translation of the RNA and alters splicing of the RNA encoding IL-18 BP, thereby reducing the yield of at least one of IL-18 BPa and IL-18 BPc.

43. The method of claim 31, further comprising administering bacillus Calmette-Guérin (BCG) to the subject in need thereof; or wherein the subject in need thereof has received or will receive BCG.

44. (canceled)

45. (canceled)

46. (canceled)

47. The method of claim 43, further comprising identifying the subject as being non-responsive to BCG monotherapy prior to administering the IL-18, the inhibitor, the antagonist, and/or the agonist.

48. The method of claim 47, wherein the inhibitor of IL-18 BP reduces the binding of IL-18 BP isoform A (IL-18 BPa) and/or IL-18 BP isoform C (IL-18 BPc) to IL-18 and/or reduces levels of IL-18 BPa and/or IL-18 BPc.

49. (canceled)

50. (canceled)

51. (canceled)

52. (canceled)

53. (canceled)

54. (canceled)

55. (canceled)

56. (canceled)

57. (canceled)

58. (canceled)

59. A method for determining treatment for bladder cancer, comprising
a) providing bladder cells from a subject with bladder cancer;
b) culturing at least a portion of the cells with IFNγ and/or culturing at least a portion of the cells with IFNγ and TNFα, each culture maintained for a period of time sufficient to induce expression of interleukin 18 binding protein (IL-18 BP) and/or IL-18 by the cells;
c) determining expression level of IL-18 BP and a gene product selected from the group consisting of i) a gene product that is expressed in urothelium and preferably not in other bladder cells, ii) a gene product for which the levels do not change when bladder cells are exposed to IFNγ, and iii) a gene product that is decreased in bladder cancer patients who do not respond to BCG therapy or who experience recurrence following BCG therapy by the cells cultured in b); and
d) determining the ratio of expression level of IL-18 BP to the gene product wherein a ratio indicative of a level of IL-18BP that is expected to interfere with BCG or IFNγ therapy indicates that the subject should receive therapy comprising an effective amount of at least one of interleukin 18 (IL-18), an inhibitor of IL-18 BP, an antagonist of an activator of IL-18 BP, and an agonist of a down-regulator of IL-18 BP or a non-BCG therapy.
